(19) United States
(12) Patent Application Publication
(10) Pub. No.: US 2026/0137352 A1
(43) Pub. Date: May 21, 2026

(54) SYSTEMS, DEVICES AND METHODS FOR GENERATING A MODEL FOR HYPERTENSION ANALYSIS USING VOICE

(71) Applicant: KVI BRAVE FUND I, INC., Toronto (CA)

(72) Inventors: Behrad TAGHIBEYGLOU, Toronto (CA); Jaycee Morgan KAUFMAN, Toronto (CA); Yan FOSSAT, Toronto (CA)

(73) Assignee: KVI BRAVE FUND I, INC., Toronto (CA)

(21) Appl. No.: 19/377,531

(22) Filed: Nov. 3, 2025

Related U.S. Application Data
(63) Continuation of application No. PCT/IB2025/055762, filed on Jun. 4, 2025.
(60) Provisional application No. 63/655,634, filed on Jun. 4, 2024.

(51) Int. Cl. A61B 5/00; G10L 25/66

(52) U.S. Cl. CPC A61B 5/7267; G10L 25/66

(57) ABSTRACT

The present embodiments are directed to computer-implemented method for generating a hypertension indicator prediction model, the method comprising: providing a plurality of voice samples from at least one subject at a plurality of time points; and a plurality of blood pressure measurements, wherein each blood pressure measurement comprises a diastolic measurement and a systolic measurement, and each blood pressure measurement is associated with a corresponding subject in the at least one subject; sorting the plurality of voice samples into two or more hypertensive categories based on the blood pressure measurement; extracting voice feature values for a set of voice features from each of the plurality of voice samples; selecting a subset of voice features from the set of voice features; and generating the hypertension indicator prediction model based on the subset of voice features.

### CROSS-REFERENCE TO RELATED APPLICATIONS

The present application claims priority to U.S. Provisional Application 63/655,634 filed Jun. 4, 2024 and PCT Patent Application PCT/IB2025/055762 the entire contents of which are incorporated herein by reference.

### FIELD

The described embodiments relate to systems, devices and methods for generating a model for providing hypertension analysis using a subject's voice and more specifically to systems, devices and methods for providing hypertension analysis including a hypertension status indicator based on voice samples.

### BACKGROUND

The following is not an admission that anything discussed below is part of the prior art or part of the common general knowledge of a person skilled in the art.

Hypertension, referred to as the “silent killer” by the World Health Organization, affects over 35% of the global population. Early diagnosis and behavioral interventions have been shown to mitigate morbidity and mortality associated with this condition. However, conventional methods of measuring blood pressure and accordingly identifying hypertension, such as sphygmomanometry, require technical expertise and may not be readily accessible, particularly in remote or underserved areas. Automatic blood pressure measurement devices offer an alternative but are often inaccessible in certain populations.

Blood pressure, defined as the pressure pushed to the walls of the blood vessels during circulation, is one of the most common indicators of cardiac health (1). The blood pressure is commonly measured at the level of arteries and varies over each heartbeat from a minimum called diastolic pressure (DBP) to a maximum called systolic pressure (SBP). The SBP is almost traceable at the beginning of the cardiac cycle and indicates the maximum pressure exerted on the walls of the arteries after contraction of the heart, while the DBP occurs in the resting phase of the cardiac cycle and shows the minimum pressure in the arteries when the heart is relaxed (2).

Hypertension is the leading risk factor for cardiovascular disorders. Early detection and initiation of treatment have been identified as the most effective ways to reduce the burden of hypertension. The most common method for detecting hypertension is blood pressure measurement, typically performed with cuff-based devices, where systolic pressure (SBP) and diastolic pressure (DBP) are measured through Korotkoff sounds. Although this method is accurate and non-invasive, it requires technical expertise and is often inaccessible in rural and remote areas.

Hypertension or the chronic elevation of resting arterial blood pressure is defined differently in different guidelines. For example, the American Health Association (AHA) has defined SBP≥130 mmHg and/or DBP≥80 mmHg, while the European Society of Hypertension (ESH) defines it as SBP≥140 mmHg and/or DBP≥90 mmHg. World Health Organization (WHO) has named hypertension a global public health concern and reports that hypertension affects 1 in 3 adults worldwide with half of them unaware of their condition, and more than 75% adults with hypertension live in low- and middle-income countries (3).

Moreover, untreated and uncontrolled hypertension increases the risk of comorbidities such as heart failure (4,5), kidney disease (6), coronary heart disease (7), and diabetes (8). Medical evidence suggests that early detection of hypertension, behavioral therapy such as changing dietary and activity habits, and tight control could reduce its further development and consequences (9).

The gold standard for blood pressure monitoring involves an invasive method, wherein a catheter is inserted directly into an artery to measure blood flow and pressure. Due to its invasive nature, this method is cumbersome and not patient friendly.

Conventionally, a non-invasive technique known as sphygmomanometry has been widely used instead. This method involves wrapping an inflatable cuff around the arm; by inflating the cuff and listening for Korotkoff sounds, SBP and DBP are measured. While sphygmomanometry is simpler and more accessible, it is not suitable for everyone, particularly individuals with sensitive artery beds or those undergoing hemodialysis. To address these limitations, alternative approaches have sought to estimate blood pressure using other biomarkers.

Other conventional approaches include methods utilizing pulse wave velocity (PWV), which can be derived from electrocardiograms (ECG) and photoplethysmograms (PPG), offer a non-invasive solution. Additionally, techniques focusing solely on PPG signals have been developed (10, 11). Although these methods provide effective solutions, they require specialized equipment and a certain level of technical expertise for accurate measurement, which make them less accessible to the general population.

Human voice is composed of complex signals that are tightly associated with physiological changes in body systems. Due to the depth of signals that can be analyzed, as well as the wide range of potential physiological dysfunction that manifest in voice signals, voice has quickly gained traction in healthcare and medical research. For example, it has been shown that thyroid hormone imbalance caused the hoarseness of voice and affected larynx development (40). Unstable pitch and loudness were observed in patients with multiple sclerosis (41). Other recent studies also demonstrated distinct voice characteristics that were associated with various pathological, neurological, and psychiatric disorders, such as congestive heart failure (42), Parkinson's disease (43), Alzheimer's disease (44), post-traumatic stress disorder (45), and autism spectrum disorder (46). The human voice is now considered as an emerging biomarker, which is inherently non-invasive, low-cost, accessible, and easy to monitor for health conditions in various real-life settings.

Voice signal analysis is an emerging non-invasive technique to examine health conditions. The analysis of human voice data (including voice signal analysis) presents a technical computer-based problem which involves digital signal processing of the voice data. Analysis, including the use of predictive models, requires significant processing capabilities in order to determine biomarker signals and extract relevant information. The sheer number of available biomarker signals poses a challenge since the biomarkers must be efficiently selected in order to reduce processing overhead. Another challenge for voice signal analysis systems performing prediction is that they preferably function in real-time with the voice data collection and on a variety of different processing platforms and operate efficiently to deliver predictions and results to a user in a timely fashion.

Recent studies have explored using speech as an accessible and straightforward modality for detecting and predicting the risk of heart failure (12-15). The underlying hypothesis is that heart failure, especially congestive heart failure, increases pulmonary edema levels, affecting the vocal cords and, consequently, speech characteristics. While hypertension is not directly linked to pulmonary edema, previous studies have investigated the association between human voice and blood pressure (16, 17), on a very limited sample size and showed some associations between hypertension and vocal characteristics. In this regard, a study by Ankicshan (18) tried classifying hypertensive and non-hypertensive speech recordings. The Ankicshan study however attempted to directly estimate the SBP and DBP values. However, the study's participant cohort lacked diversity in terms of gender and the range of blood pressure measurements. Moreover, their reported validations were based on K-fold cross-validation, which does not ensure participants were segmented in a manner where all their data remained within the same fold. As a result, there is high possibility of data leakage, resulting in limited generalizability of their findings to other cohorts.

There remains a need for systems and methods for hypertension tracking applications for subjects that provide low-cost and accessible generation of hypertension indicators about subjects using voice.

### SUMMARY

The following summary is provided to introduce the reader to the more detailed discussion to follow. The summary is not intended to limit or define any claimed or as yet unclaimed invention. One or more inventions may reside in any combination or sub-combination of the elements or process steps disclosed in any part of this document including its claims and figures.

Provided are systems, devices and methods for providing a hypertension indicator for a subject and associated embodiments.

In Example #1, a framework for detecting hypertension through acoustic analysis of speech was evaluated. By recording speech across multiple sessions and analyzing its temporal and spectral characteristics, indicators of hypertension were explored. Two thresholds for labeling individuals with hypertension were used: (I) systolic blood pressure (SBP)≥135 mmHg or diastolic blood pressure (DBP)≥85 mmHg, and (II) SBP≥140 mmHg or DBP≥90 mmHg. Example #1 involved 245 participants, including 91 females. Predictive models were developed for each gender and their performance was assessed using leave-one-subject-out validation. For the first threshold, the balanced accuracy achieved was 84% for females and 77% for males. For the second threshold, the corresponding balanced accuracies were 63% for females and 86% for males. These results demonstrate the potential of utilizing speech-based representations for non-invasive screening of hypertension.

Example #1 analyzed acoustic speech features from recordings of acoustic speech features from recordings of 245 participants, classifying them according to two thresholding criteria. Feature selection techniques were utilized and integrated 12 different classifiers for each gender to enhance the robustness and accuracy of our analyses.

In Example #2, the feasibility of using covert speech (random speech corpora) for hypertension screening was investigated through multiple short recordings for hypertension screening based on two hypertension guidelines: 1) SBP≥135 mmHg OR DBP≥85 mmHg, and 2) SBP≥140 mmHg OR DBP≥90 mmHg. This example incorporated speech recordings from 573 participants, including 197 females, with diverse ages and body-mass-index (BMI) and extracted temporal, spectral, and non-linear acoustic features through three different frameworks, all based on classical and boosted machine learning models. The models were evaluated using a leave-one-subject-out (LOSO) cross-validation scheme. For the first threshold (SBP≥135 OR DBP≥85), the balanced accuracy achieved was 70% for females and 61% for males. For the second threshold (SBP≥140 OR DBP≥90), the corresponding balanced accuracies were 78% for females and 71% for males.

Moreover, a leave-one-subject-out validation method was employed to report findings, ensuring a thorough and individualized assessment of the model's performance across all participants.

In a first aspect there is provided a computer-implemented method for providing a hypertension indicator for a subject, the method comprising: providing, at a memory, a hypertension indicator prediction model; receiving, at a processor in communication with the memory, a voice sample from the subject; extracting, at the processor, at least one voice biomarker feature value from the voice sample for at least one predetermined voice biomarker feature; determining, at the processor, the hypertension indicator for the subject based on the at least one voice biomarker feature value and the hypertension indicator prediction model; and outputting, at an output device, the hypertension indicator for the subject or an output based on the hypertension indicator.

In one or more embodiments, the hypertension indicator for the subject may be one of a plurality of categories.

In one or more embodiments, the hypertension indicator for the subject may be a category based on the European Society of Hypertension (ESH) hypertension guideline.

In one or more embodiments, the plurality of categories may comprise: a first hypertensive category comprising SBP>=135 OR DBP>=85; and a non-hypertensive category.

In one or more embodiments, the plurality of categories may comprise: a second hypertensive category comprising SBP>=140 OR DBP>=90.

In one or more embodiments: the at least one predetermined voice biomarker feature may be selected from the group of a non-linear feature, a low-level descriptor feature, a time related feature, and a frequency-related feature.

In one or more embodiments, the non-linear feature may comprise one of a Shannon entropy of the voice sample and a logarithm entropy of the voice sample, the low-level descriptor feature may comprise one of a mean of the voice sample, a variance of the voice sample, a skewness of the voice sample, a kurtosis of the voice sample, a range of the voice sample, a cumulative sum of the first derivative (CSFD) of the signal, the time related feature may comprise one of a mean of the zero-crossing rate, a standard deviation of the zero-crossing rate, and the frequency-related feature may comprise one of a mean Mel-scale frequency cepstral coefficient (MFCC), a standard deviation of a Mel-scale frequency cepstral coefficient (MFCC), a first (ΔMFCC) and a second derivative (ΔΔMFCC) of the MFCC, a mean Chromagram coefficient, a standard deviation of a Chromagram coefficient, a mean spectral contrast, a standard deviation of spectral contrast, a centroid of a spectral contrast, a bandwidth of a spectral contrast, a flatness of a spectral contrast, a roll-off of a spectral contrast, a fundamental frequency, an LLD of a tempogram, a power spectral density (PSD) from 0 to

$$\frac{{F}_{s}}{2} Hz ,$$

an LLD of the PSD, an entropies of the PSD, and a coefficient of a polynomial curve fitted to the PSD.

In one or more embodiments, wherein the hypertension indicator prediction model may comprise a statistical classifier.

In one or more embodiments, the statistical classifier may comprise at least one selected from the group of: a support vector classifier (SVC), a K-nearest neighbor (KNN) classifier, a logistic regression (LR) classifier, a linear discriminant analysis (LDA) classifier, quadratic discriminant analysis (QDA) classifier, a Gaussian naïve Bayesian (GNB) classifier, Bernoulli naïve Bayesian (BNB) classifier, Ridge, an extra gradient boosting (XGBoost) classifier, decision tree (DT) classifier, and random forest (RF) classifier.

In one or more embodiments: the hypertension indicator prediction model may be an ensemble model, the ensemble model comprising n LDA classifiers; and wherein the determining, at the processor, the hypertension indicator may comprise: determining a prediction from each of the n LDA classifiers in the ensemble model; and determining the hypertension indicator based on an election of the predictions from the n LDA classifiers in the ensemble model.

In one or more embodiments, the method may further comprise: transmitting, to a user device in network communication with the processor, the hypertension indicator for the subject, wherein the outputting of the hypertension indicator for the subject occurs at the user device.

In one or more embodiments, the method may further comprise: determining the hypertension indicator for the subject based on at least one clinicopathological value for the subject, optionally at least one of height, weight, BMI, diabetes status and blood pressure.

In one or more embodiments, the voice sample may comprise a predetermined phrase vocalized by the subject.

In one or more embodiments, the voice sample may comprise a randomly selected phrase vocalized by the at least one subject.

In one or more embodiments, the predetermined phrase or the randomly selected phrase may be displayed to the subject on the user device.

In one or more embodiments, the voice sample may be received from an audio sensor at the user device.

In a second aspect there is provided a system for determining a hypertension indicator for a subject, the system comprising a memory and a processor configured to operate the methods described herein.

In a third aspect, there is provided a device for determining a hypertension indicator for a subject, the device comprising a memory and a processor configured to operate the methods described herein.

In a fourth aspect, there is provided a computer-implemented method for generating a hypertension indicator prediction model, the method comprising: providing, at a memory: a plurality of voice samples from at least one subject at a plurality of time points; and a plurality of blood pressure measurements, wherein each blood pressure measurement comprises a diastolic measurement and a systolic measurement, and each blood pressure measurement is associated with a corresponding subject in the at least one subject; sorting, at a processor in communication with the memory, the plurality of voice samples into two or more hypertensive categories based on the blood pressure measurement; extracting, at the processor, voice feature values for a set of voice features from each of the plurality of voice samples; selecting, at the processor, a subset of voice features from the set of voice features; and generating at the processor, the hypertension indicator prediction model based on the subset of voice features.

In one or more embodiments, generating the hypertension indicator prediction model based on the subset of voice features may comprise determining a weight for each voice feature in the subset of voice features.

In one or more embodiments, the method may further comprise: selecting, at the processor, a subset of voice features from the set of voice features based on a Least Absolute Shrinkage and Selection Operator (LASSO) method.

In one or more embodiments, the LASSO objective function may be

$${}_{β}^{m i n} \frac{1}{2 N} {‖ X β - y ‖}_{2}^{2} + α {‖ β ‖}_{1} .$$

In one or more embodiments, each of the voice features may be selected from the group of a non-linear feature, a low-level descriptor feature, a time related feature, and a frequency-related feature.

In one or more embodiments: the non-linear feature may comprise one of a Shannon entropy of the voice sample and a logarithm entropy of the voice sample, the low-level descriptor feature may comprise one of a mean of the voice sample, a variance of the voice sample, a skewness of the voice sample, a kurtosis of the voice sample, a range of the voice sample, a cumulative sum of the first derivative (CSFD) of the signal, the time related feature may comprise one of a mean of the zero-crossing rate, a standard deviation of the zero-crossing rate, and the frequency-related feature may comprise one of a mean Mel-scale frequency cepstral coefficient (MFCC), a standard deviation of a Mel-scale frequency cepstral coefficient (MFCC), a first (ΔMFCC) and a second derivative (ΔΔMFCC) of the MFCC, a mean Chromagram coefficient, a standard deviation of a Chromagram coefficient, a mean spectral contrast, a standard deviation of spectral contrast, a centroid of a spectral contrast, a bandwidth of a spectral contrast, a flatness of a spectral contrast, a roll-off of a spectral contrast, a fundamental frequency, an LLD of a tempogram, a power spectral density (PSD) from 0 to

$$\frac{{F}_{s}}{2} Hz ,$$

an LLD of the PSD, an entropies of the PSD, and a coefficient of a polynomial curve fitted to the PSD.

In one or more embodiments, the method may further comprise: dividing, at the processor, each of the plurality of voice samples a plurality of equidistant segments; extracting, at the processor, voice feature values for a set of voice features from each of the plurality of equidistant segments; and wherein the voice feature values for each voice sample may comprise a concatenation of the voice feature values for the corresponding plurality of equidistant segments.

In one or more embodiments, the method may further comprise: averaging, at the processor, at least two voice samples in the plurality of voice samples, the at least two voice samples corresponding to the same subject.

In one or more embodiments, the method may further comprise: applying, at the processor, a synthetic minority oversampling technique (SMOTE) to increase a number of the plurality of voice samples.

In one or more embodiments, the method may further comprise: determining, at the processor, a true positive rate (TPR) and a true negative rate (TNR) for the generated hypertension indicator prediction model using a leave-one-subject-out (LOSO) method.

In one or more embodiments, the two or more hypertensive categories may comprise a category based on European Society of Hypertension (ESH) hypertension guidelines.

In one or more embodiments, the two or more hypertensive categories may comprise at least a first hypertensive category comprising SBP>=135 OR DBP>=85 and a second hypertensive category comprising SBP>=140 OR DBP>=90.

In one or more embodiments, the voice sample may comprise a predetermined phrase vocalized by the at least one subject.

In one or more embodiments, the voice sample may comprise a randomly selected phrase vocalized by the at least one subject.

In a fifth aspect, there is provided a system for generating a hypertension indicator prediction model, the system comprising a memory and a processor, the processor configured to provide the methods described herein.

It will be appreciated by a person skilled in the art that a system, device, method or computer program product disclosed herein may embody any one or more of the features contained herein and that the features may be used in any particular combination or sub-combination. Other features and advantages of the present application will become apparent from the following detailed description taken together with the accompanying drawings. It should be understood, however, that the detailed description and the specific examples are given by way of illustration only, since various changes and modifications within the scope of the application will become apparent to those skilled in the art from this detailed description.

### BRIEF DESCRIPTION OF THE DIAGRAMS

For a better understanding of the various examples described herein, and to show more clearly how these various examples may be carried into effect, reference will be made, by way of example, to the accompanying drawings which show at least one example, and which are now described. The drawings are not intended to limit the scope of the teachings described herein.

A preferred embodiment of the present invention will now be described in detail with reference to the diagrams, in which:

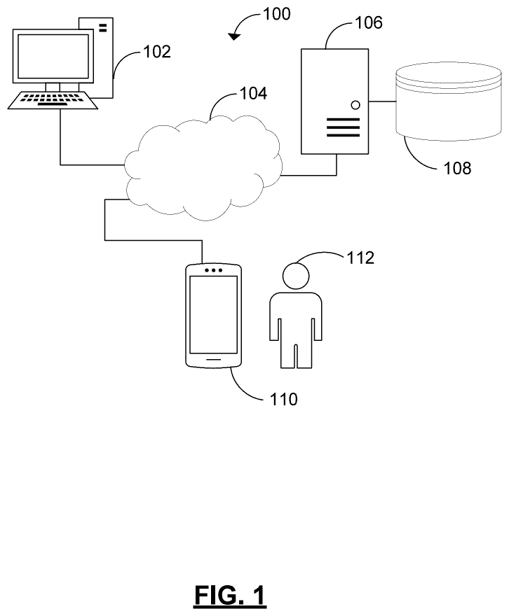

FIG. 1 shows a system diagram in accordance with one or more embodiments.

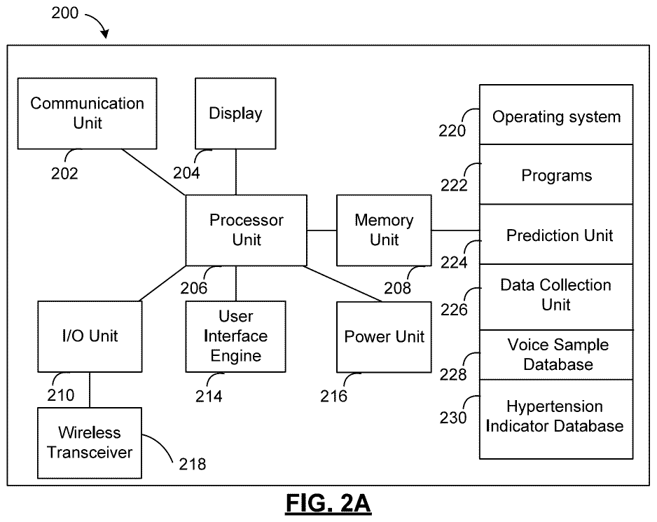

FIG. 2A shows a device diagram in accordance with one or more embodiments.

FIG. 2B shows a device diagram in accordance with one or more embodiments.

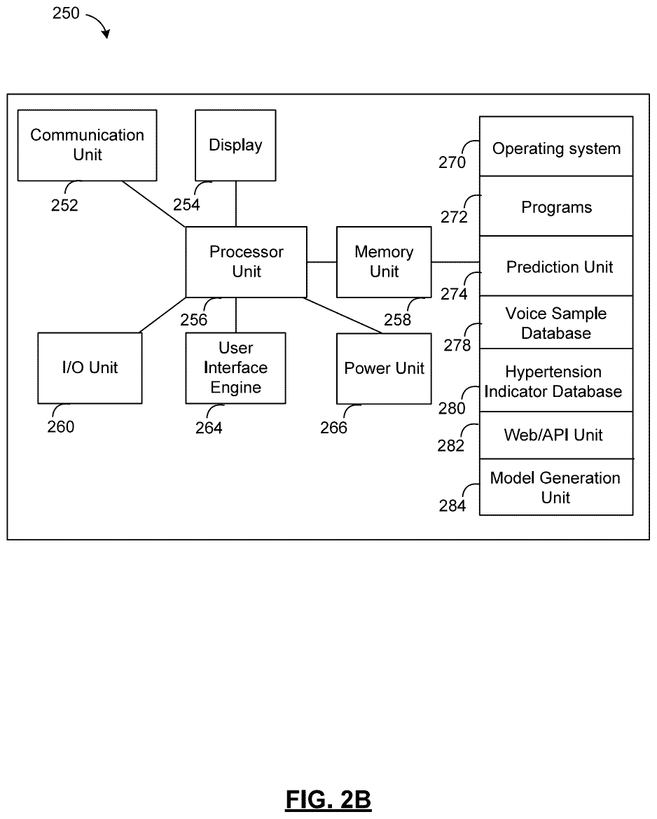

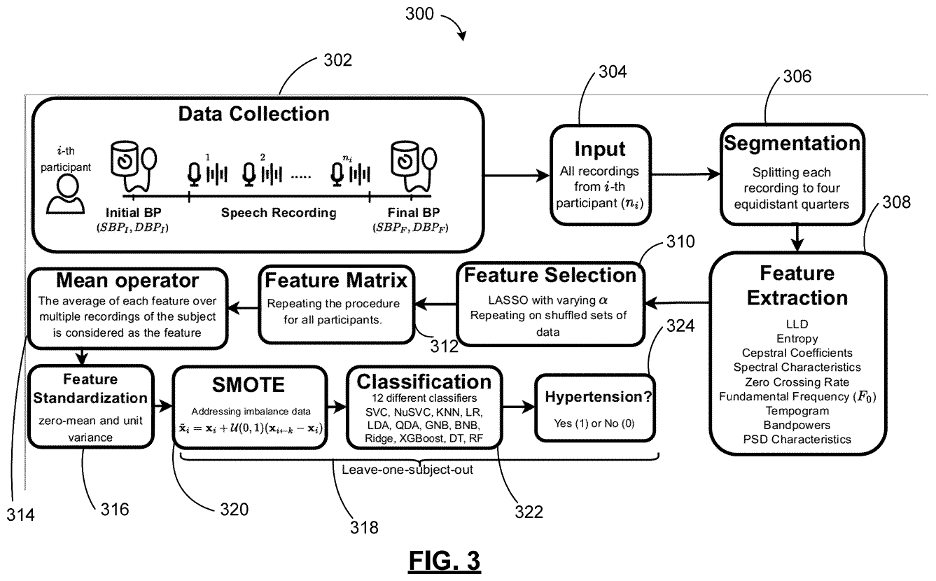

FIG. 3 shows a model development diagram in accordance with one or more embodiments.

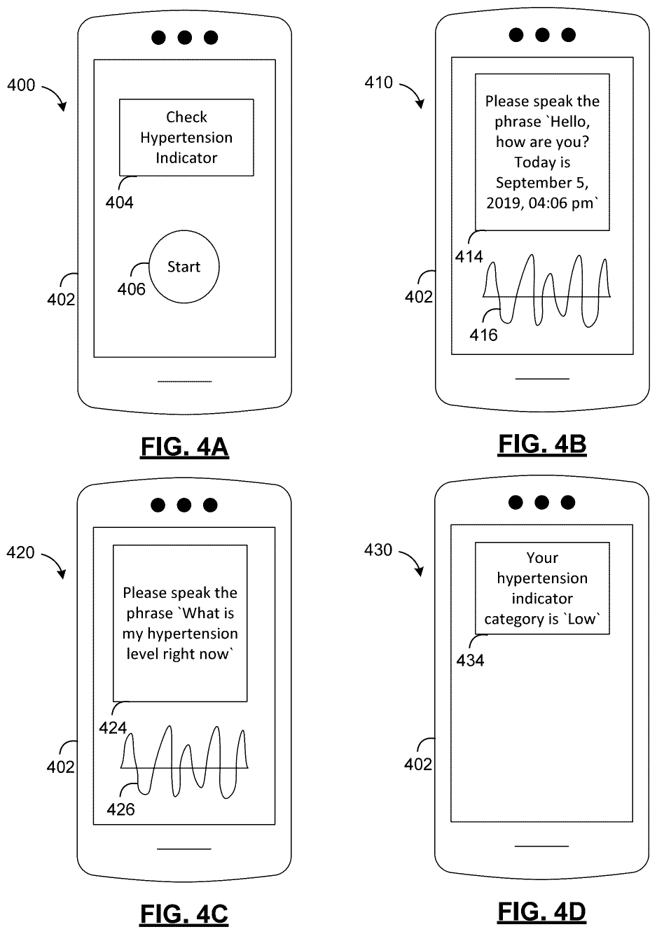

FIGS. 4A, 4B, 4C, and 4D show user interface diagrams in accordance with one or more embodiments.

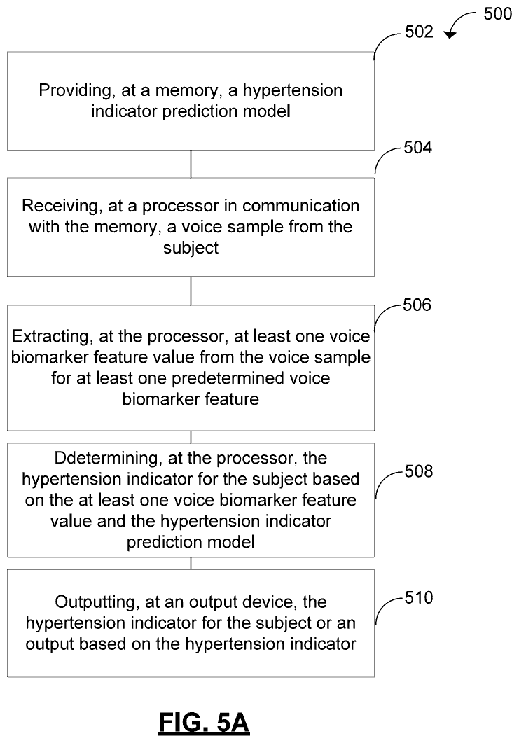

FIG. 5A shows a computer-implemented method diagram in accordance with one or more embodiments.

FIG. 5B shows another computer-implemented method diagram in accordance with one or more embodiments.

FIG. 6 shows a table of the list of extracted features in Example #1. s[n], Pₛ, and Pₛ[f₁, f₂] are N-sample signal s, power spectrum of s[n], and normalized band power of s[n] in the frequency band between f₁ and f₂ (in Hz), which can be denoted as

$$\frac{{\int }_{{f}_{1}}^{{f}_{2}} {P}_{s} df}{{\int }_{0}^{{f}_{s}} {P}_{s} df}$$

respectively. The CDS is also defined as

$${\sum }_{n = 1}^{N} s [ n ] - s [ n - 1 ] .$$

μₜ and σₜ are mean and standard deviation operators, respectively, that are applied through the output time frames of the target feature.

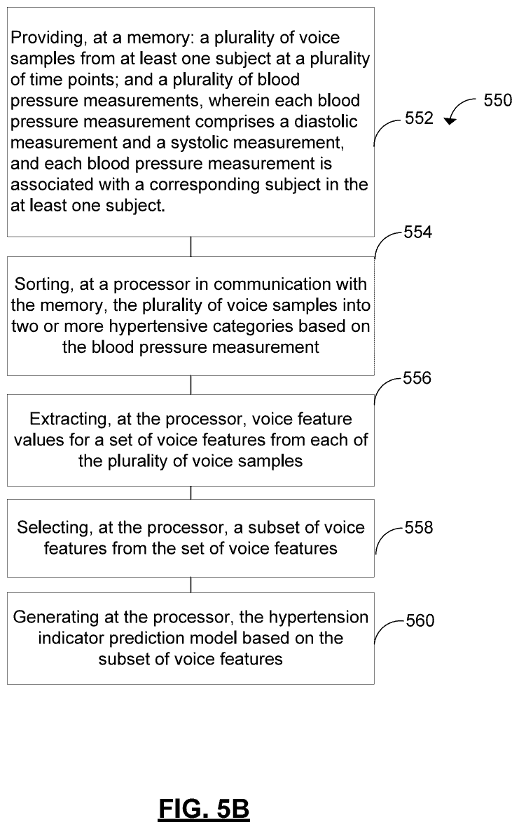

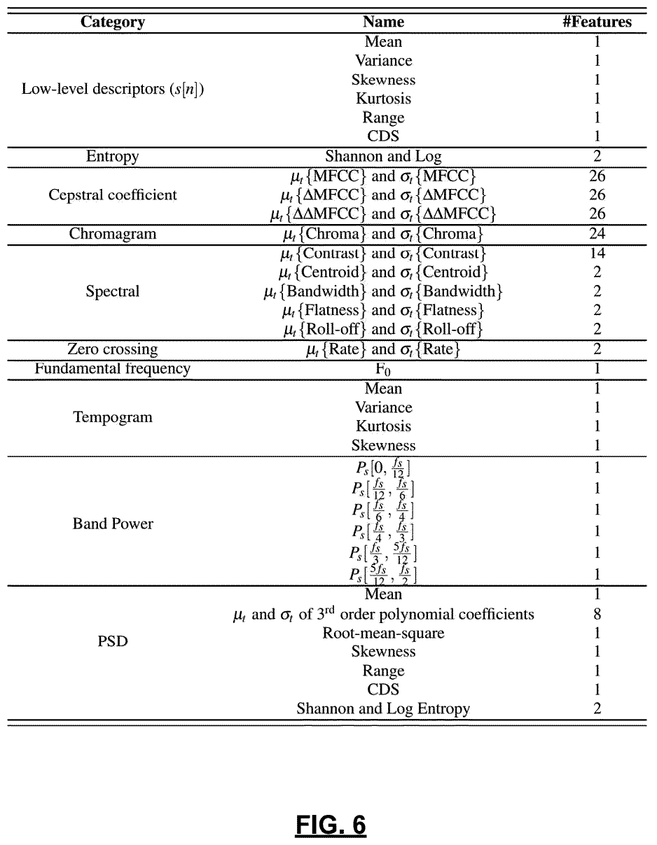

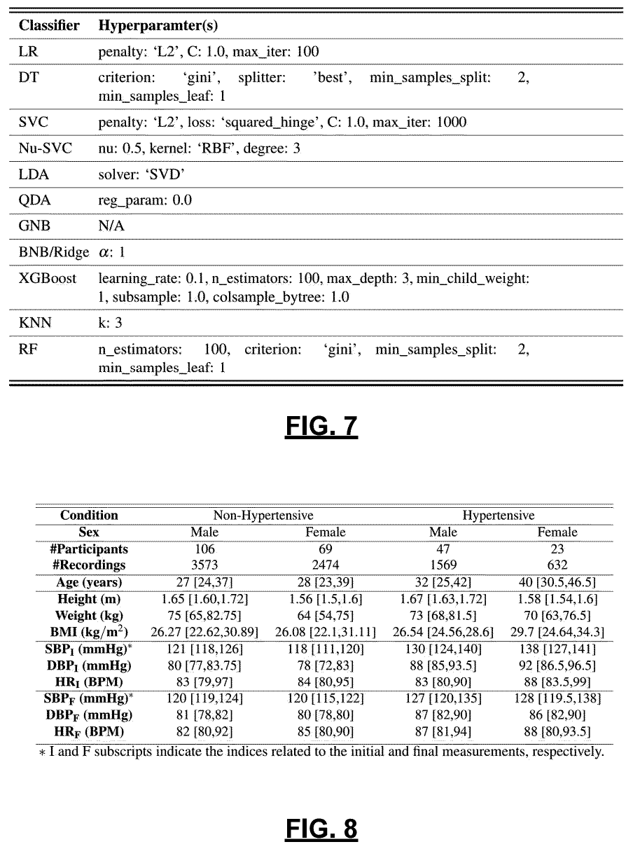

FIG. 7 shows a table of the details of classifiers used in the proposed method in Example #1.

FIG. 8 shows a demographic table of the dataset of Example #1 based on SBP≥135 mmHg or DBP≥85 mmHg for annotating hypertension. All continuous variables are reported by their first three quartiles (Q2 [Q1,Q3]). SBP, DBP, HR, and BPM indicate systolic and diastolic blood pressure, heart rate, and beats per minute, respectively.

FIG. 9 shows a demographic table of the dataset of Example #1 based on SBP≥140 mmHg or DBP≥90 mmHg for annotating hypertension. All continuous variables are reported by their first three quartiles (Q2 [Q1,Q3]). SBP, DBP, HR, and BPM indicate systolic and diastolic blood pressure, heart rate, and beats per minute, respectively.

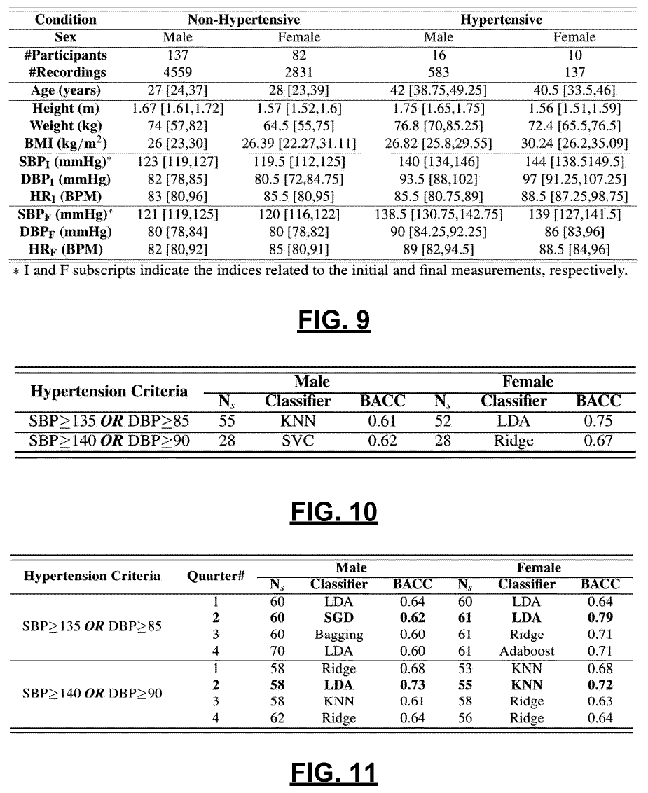

FIG. 10 shows a result table from Example #1 including the result of full recording length pipeline. Nₛ is the number of selected features.

FIG. 11 shows a quarter-based results table from Example #1 including quarter-based results. Nₛ is the number of selected features and the best quarter in terms of classification performance is highlighted in bold font.

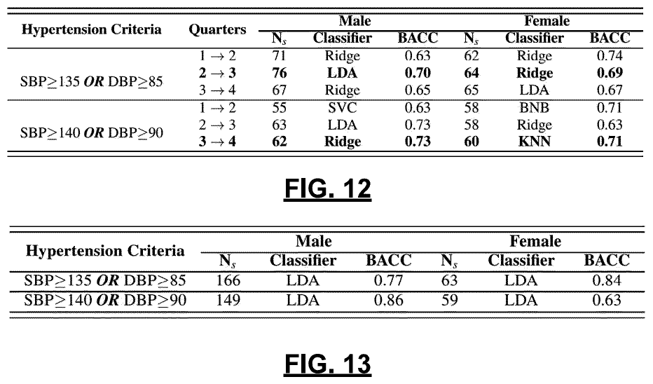

FIG. 12 shows a cross-quarter results table from Example #1 including cross-quarter results. Nₛ is the number of selected features and the best-performed model for each hypertension criterion is highlighted in bold font.

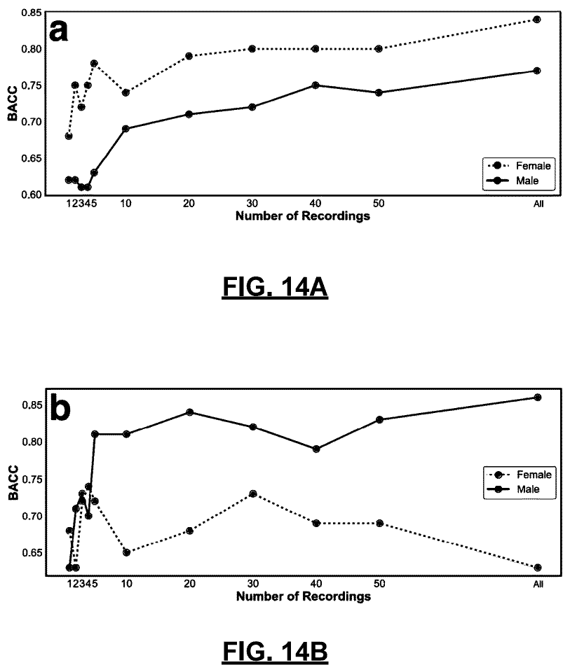

FIG. 13 shows a stacked quarter result table from Example #1 including shown stacked quarters results. Nₛ is the number of selected features.

FIG. 14A shows a results table from Example #1 of the effect of recordings numbers used for evaluating the performance of the proposed models under a first threshold SBP≥135 OR DBP≥85.

FIG. 14B shows a results table from Example #1 of the effect of recordings numbers used for evaluating the performance of the proposed models under a second threshold SBP≥140 OR DBP≥90.

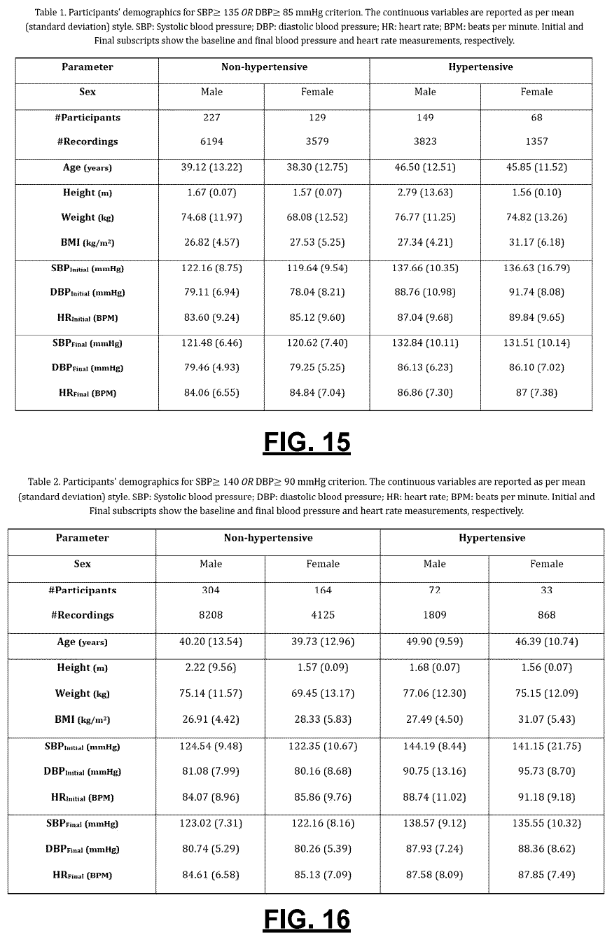

FIG. 15 shows a participant demographic table of the dataset of Example #2 based on SBP≥135 mmHg or DBP≥85 mmHg criterion. The continuous variables are reported as per mean (standard deviation) style. SBP: Systolic blood pressure; DBP: diastolic blood pressure; HR: heart rate; BPM: beats per minute. Initial and Final subscripts show the baseline and final blood pressure and heart rate measurements, respectively.

FIG. 16 shows a demographic table of the dataset of Example #2 based on SBP≥140 mmHg or DBP≥90 mmHg for annotating hypertension. The continuous variables are reported as per mean (standard deviation) style. SBP: Systolic blood pressure; DBP: diastolic blood pressure; HR: heart rate; BPM: beats per minute. Initial and Final subscripts show the baseline and final blood pressure and heart rate measurements, respectively.

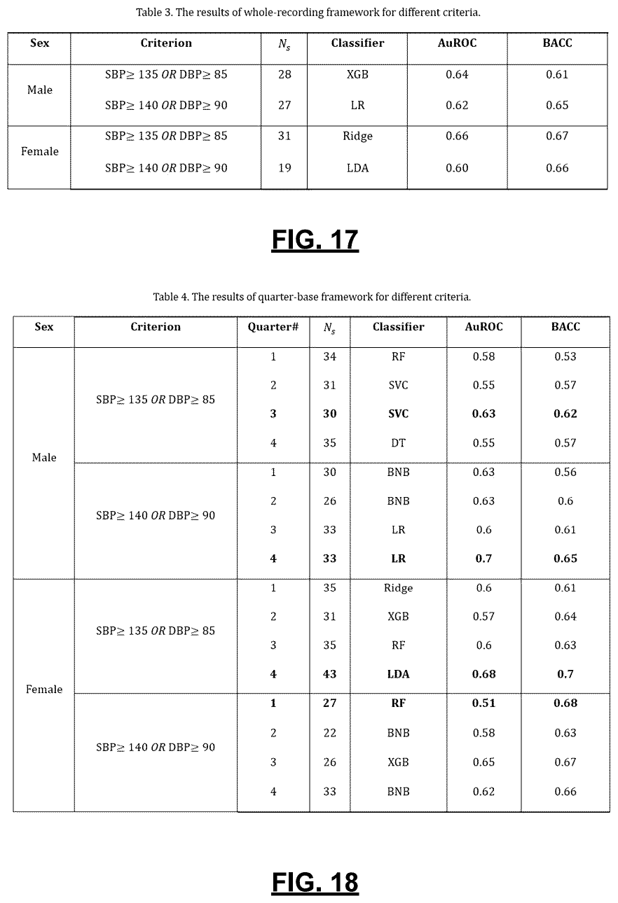

FIG. 17 shows a whole-recording framework result from Example #2 for different criteria.

FIG. 18 shows a quarter-based framework result from Example #2 for different criteria.

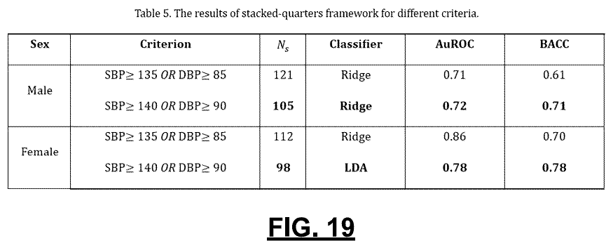

FIG. 19 shows a stacked quarter framework result from Example #2 for different criteria.

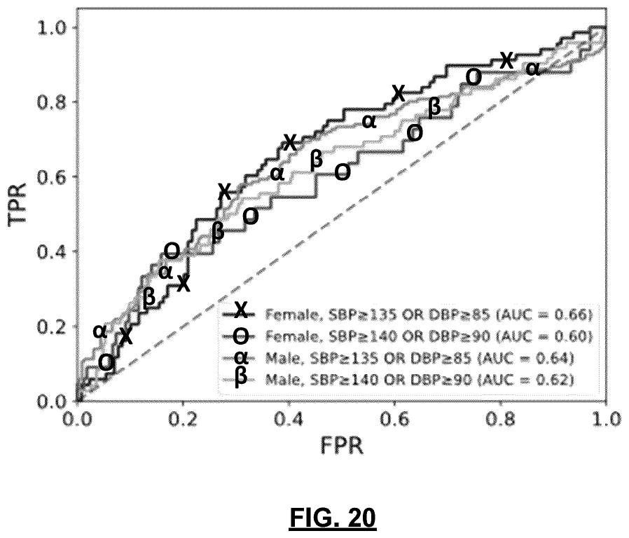

FIG. 20 shows a Whole Recording ROC diagram, including AUC: area under curve; TPR: true positive rate; FPR: false positive rate for Example #2.

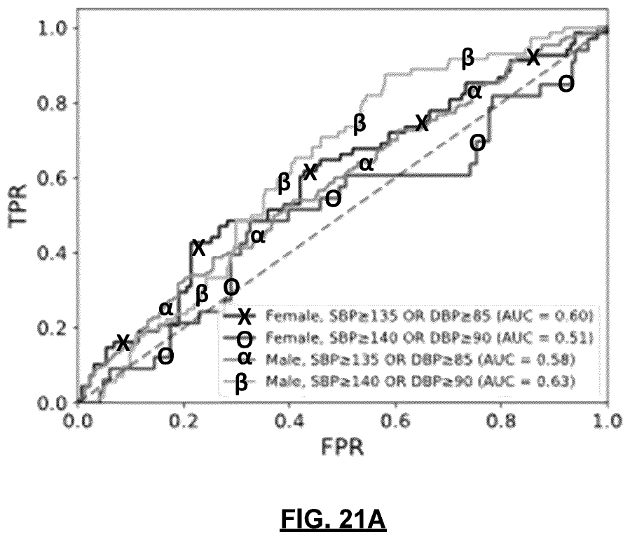

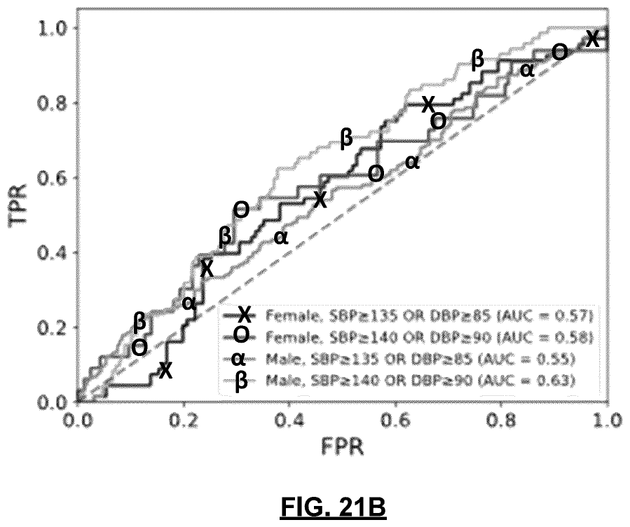

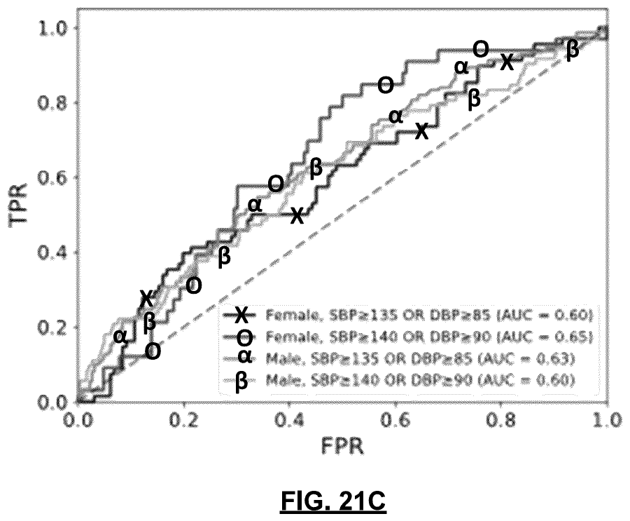

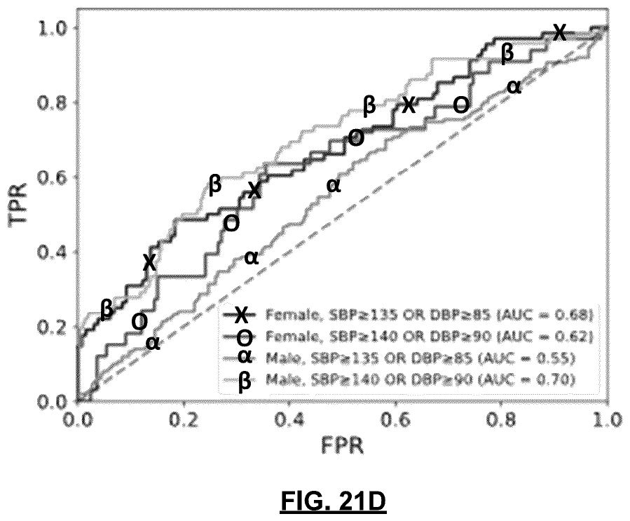

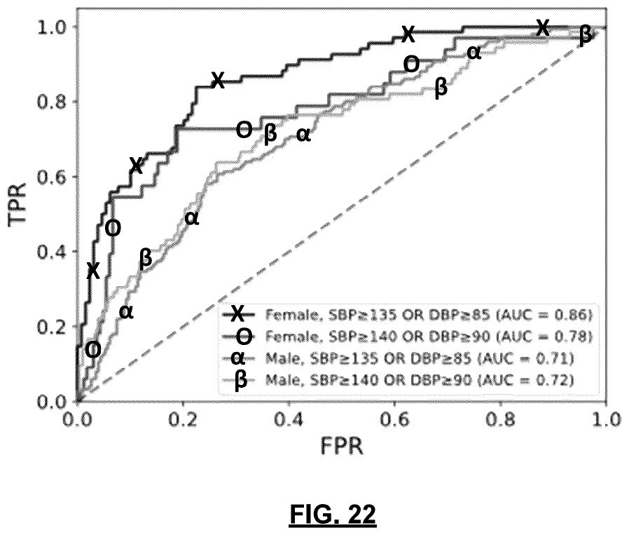

FIGS. 21A-21D show a Quarter ROC diagram, including AUC: area under curve; TPR: true positive rate; FPR: false positive rate for Example #2. FIG. 21A shows the ROC diagram for the first equidistant quarter. FIG. 21B shows the ROC diagram for the second equidistant quarter. FIG. 21C shows the ROC diagram for the third equidistant quarter. FIG. 21D shows the ROC diagram for the fourth equidistant quarter.

FIG. 22 shows a Stacked quarter ROC, including AUC: area under curve; TPR: true positive rate; FPR: false positive rate for Example #2.

### DESCRIPTION OF EXEMPLARY EMBODIMENTS

Various embodiments will now be described below to provide an example of the claimed subject matter. No example described below limits any claimed subject matter and any claimed subject matter may cover embodiments such as systems or methods that differ from those described below.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures, and components have not been described in detail so as not to obscure the examples described herein. Also, the description is not to be considered as limiting the scope of the examples described herein.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g., 112a, or 112₁). Multiple elements herein may be identified by part numbers that share a base number in common and that differ by their suffixes (e.g., 112₁, 112₂, and 112₃). All elements with a common base number may be referred to collectively or generically using the base number without a suffix (e.g., 112).

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g., a keyboard, a mouse, a touchscreen, and the like), and at least one output device (e.g., a display screen, a printer, a wireless radio, and the like) depending on the nature of the device. For example, and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g., ROM, magnetic disk, optical disc) readable by a general or special purpose programmable computer, for configuring and operating the computer when the storage media or device is read by the computer to perform the procedures described herein. Examples of the system may also be considered to be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

As used herein, the term “user” refers to a user of a user device, and the term “subject” refers to a subject whose measurements are being collected. The user and the subject may be the same person, or they may be different persons in the case where one individual operates the user device and another individual is the subject. For example, in one embodiment the user may be a health care professional such as a nurse or doctor and the subject is a human patient.

Referring first to FIG. 1, there is shown a system diagram 100 for a system for determining a hypertension indicator for a subject and a system for generating a hypertension indicator prediction model.

System 100 includes one or more computer devices 102, a network 104, one or more servers 106, one or more data stores 108, and one or more user devices 110 for one or more users 112.

The computer-implemented prediction system performs voice analysis which may be used to predict and identify a hypertension indicator for a subject. The prediction system may identify a hypertension category for the subject, a risk score, or another type of indicator that may be communicated to the subject or user. Optionally, the voice recording may be made at a regular interval, for example, weekly or daily.

The interval recordings may be collected generally at the same time every day. A software application running on the user device 110 may provide one or more notifications, including notifications related to hypertension for a user 112 of the user device 110 (including where the user is the subject of the measurements). The notifications may remind the user to collect voice samples from the subject (themselves, in the case where the user and the subject are the same individual).

The one or more computer devices 102 may be used by a clinician user such as an administrator, clinician, cardiologist, or other medical professional to access a software application (not shown) running on server 106 over network 104. In one embodiment, the one or more computer devices 102 may access a web application hosted at server 106 using a browser for reviewing hypertension indicators given to the users 112 (including users who are subjects) using user devices 110.

The one or more user devices 110 may download an application (including downloading from an App Store such as the Apple® App Store or the Google® Play Store) for determining hypertension indicators for the users 112 (including subjects who are users) using user devices 116.

The one or more user devices 110 may be any two-way communication device with capabilities to communicate with other devices. A user device 110 may be a mobile device such as mobile devices running the Google® Android® operating system or Apple® iOS® operating system. A user device 110 may be a smart speaker, such as an Amazon® Alexa® device, or a Google® Home® device. A user device 110 may be a smart watch such as the Apple® Watch, Samsung® Galaxy® watch, a Fitbit® device, or others as known. A user device 110 may be a purpose-built sensor system attached to the body of, or on the clothing of, a user.

A user device 110 may be the personal device of a user or may be a device provided by an employer. The one or more user devices 110 may be used by an end user 112 to access the software application (not shown) running on server 106 over network 104. In one embodiment, the one or more user devices 110 may access a web application hosted at server 106 using a browser for determining hypertension indicators. In an alternate embodiment, the one or more user devices 110 may download an application (including downloading from an App Store such as the Apple® App Store or the Google® Play Store) for determining hypertension indicators. The user device 110 may be a desktop computer, mobile device, or laptop computer. The user device 110 may be in communication with server 106 and may allow a user 112 to review a user profile stored in a database at data store 108, including historical hypertension indicators. The users 112 using user devices 110 may provide one or more voice samples using a software application and may receive a hypertension indicator based on the one or more voice samples as described herein.

The one or more user devices 110 may each have one or more audio sensors. The one or more audio sensors may be in an array. The audio sensors may be used by a user 112 of the software application to record a voice sample into the memory of the user device 110. The one or more audio sensors may be an electret microphone onboard the user device, MEMS microphone onboard the user device, a Bluetooth enabled connection to a wireless microphone, a line in, etc.

The software application running on the one or more user devices 110 may communicate with server 106 using an Application Programming Interface (API) endpoint, and may send and receive voice sample data, user data, mobile device data, and mobile device metadata.

The software application running on the one or more user devices 110 may display one or more user interfaces on a display device of the user device, including, but not limited to, the user interfaces shown in FIGS. 4A, 4B, 4C, and 4D.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The server 106 is in network communication with the one or more user devices 110 and the one or more computer devices 102. The server 106 may further be in communication with a database at data store 108. The database at data store 108 and the server 106 may be provided on the same server device, may be configured as virtual machines, or may be configured as containers. The server 106 and a database at data store 108 may run on a cloud provider such as Amazon® Web Services (AWS®).

The server 106 may host a web application or an Application Programming Interface (API) endpoint that the one or more user devices 110 may interact with via network 104. The server 106 may make calls to the mobile device 110 to poll for voice sample data. Further, the server 106 may make calls to the database at data store 108 to query subject data, voice sample data, hypertension data, or other data received from the users 112 of the one or more user devices 110. The requests made to the API endpoint of server 106 may be made in a variety of different formats, such as JavaScript Object Notation (JSON) or eXtensible Markup Language (XML). The voice sample data may be transmitted between the server 106 and the user device 110 in a variety of different formats, including MP3, MP4, AAC, WAV, Ogg Vorbis, FLAC, or other audio data formats as known. The voice sample data may be stored as Pulse-Code Modulation (PCM) data. The voice sample data may be recorded at 22,050 Hz or 44,100 Hz. The voice sample data may be collected as a mono signal, or a stereo signal. The voice sample data may be encrypted by user device 110 prior to transmission to server 106. The voice sample data received by the data store 108 from the one or more user devices 110 may be stored in the database at data store 108 or may be stored in a file system at data store 108. The file system may be a redundant storage device at the data store 108 or may be another service such as Amazon® S3, or Dropbox.

The database of data store 108 may store subject information including hypertension data, subject and/or user information including subject and/or user profile information, and configuration information. The database of data store 108 may be a Structured Query Language (SQL) such as PostgreSQL or MySQL or a not only SQL (NoSQL) database such as MongoDB.

Referring next to FIG. 2A, there is shown a device diagram 200 in accordance with one or more embodiments. The user device diagram 200 shows detail of the one or more user devices 110 in FIG. 1. The user device 200 includes one or more of a communication unit 202, a display 204, a processor unit 206, a memory unit 208, I/O unit 210, a user interface engine 214, a power unit 216, and a wireless transceiver 218. The user device 200 may be a laptop, gaming system, smart speaker device, mobile phone device, smart watch or others as are known. The user device 200 may be a passive sensor system proximate to the user, for example, a device worn on user, or on the clothing of the user.

The communication unit 202 can include wired or wireless connection capabilities. The communication unit 202 can include a radio that communicates utilizing CDMA, GSM, GPRS or Bluetooth protocol according to standards such as IEEE 802.11a, 802.11b, 802.11g, or 802.11n. The communication unit 202 can be used by the mobile device 200 to communicate with other devices or computers.

Communication unit 202 may communicate with the wireless transceiver 218 to transmit and receive information via a local wireless network with a microphone. In an alternate embodiment, the communication unit 202 may communicate with the wireless transceiver 218 to transmit and receive information via local wireless network with an optional handheld device associated with the hypertension prediction device 200. The communication unit 202 may provide communications over the local wireless network using a protocol such as Bluetooth (BT) or Bluetooth Low Energy (BLE).

The display 204 may be an LED or LCD based display and may be a touch sensitive user input device that supports gestures.

The processor unit 206 controls the operation of the mobile device 200. The processor unit 206 can be any suitable processor, controller or digital signal processor that can provide sufficient processing power depending on the configuration, purposes and requirements of the user device 200 as is known by those skilled in the art. For example, the processor unit 206 may be a high-performance general processor. In alternative embodiments, the processor unit 206 can include more than one processor with each processor being configured to perform different dedicated tasks. In alternative embodiments, it may be possible to use specialized hardware to provide some of the functions provided by the processor unit 206. For example, the processor unit 206 may include a standard processor, such as an Intel® processor, an ARM® processor or a microcontroller.

The processor unit 206 can also execute a user interface (UI) engine 214 that is used to generate various UIs, some examples of which are shown and described herein, such as interfaces shown in FIGS. 4A-4D.

The present systems, devices and methods may provide an improvement in the operation of the processor unit 206 by ensuring the analysis of voice data and hypertension indicators are made using relevant biomarkers. The reduced processing required for the relevant biomarkers in the analysis (as compared with processing the superset of all biomarkers) reduces the processing burden required to make hypertension indicators available based on voice data.

The memory unit 208 comprises software code for implementing an operating system 220, programs 222, prediction unit 224, data collection unit 226, voice sample database 228, and hypertension indicator database 230.

The present systems and methods may provide an improvement in the operation of the memory unit 208 by ensuring the analysis of voice data is performed using relevant biomarkers and thus only relevant biomarker data is stored. The reduced storage required for the relevant biomarkers in the analysis (as compared with processing the superset of all biomarkers) reduces the memory overhead required to make hypertension predictions based on voice data.

The memory unit 208 can include RAM, ROM, one or more hard drives, one or more flash drives or some other suitable data storage elements such as disk drives, etc. The memory unit 208 is used to store an operating system 220 and programs 222 as is commonly known by those skilled in the art.

The I/O unit 210 can include at least one of a mouse, a keyboard, a touch screen, a thumbwheel, a track-pad, a track-ball, a card-reader, an audio source, a microphone, voice recognition software and the like again depending on the particular implementation of the user device 200. In some cases, some of these components can be integrated with one another.

The user interface engine 214 is configured to generate interfaces for users to configure voice measurement, connect to a hypertension monitoring device or audio input device, record voice data, view hypertension predictions, view voice sample data, etc. The various interfaces generated by the user interface engine 214 are displayed to the user on display 204.

The power unit 216 can be any suitable power source that provides power to the user device 200 such as a power adaptor or a rechargeable battery pack depending on the implementation of the user device 200 as is known by those skilled in the art.

The operating system 220 may provide various basic operational processes for the user device 200. For example, the operating system 220 may be a mobile operating system such as Google® Android® operating system, or Apple® iOS® operating system, or another operating system.

The programs 222 include various user programs so that a user can interact with the user device 200 to perform various functions such as, but not limited to, viewing hypertension indictor data, voice data, recording voice samples, receiving and viewing hypertension indicator data from a device worn on or about the subject, receiving any other data related to hypertension predictions, as well as receiving messages, notifications and alarms as the case may be. The programs 222 may be downloaded from an application store (“app store”) such as the Apple® App Store® or the Google® Play Store®.

In one or more embodiments, the programs 222 may include a hypertension tracking application. The hypertension tracking application may record voice samples from the user and report the subject's hypertension indicators. Such a hypertension application may integrate with a health tracker of the individual such as a Fitbit®, or Apple® Watch such that additional measurement data may be collected. The hypertension application may record historical hypertension indicators in order to determine changes in the user's hypertension indicators. The embodiments described herein may allow for a user to check their relative hypertension indicator using voice samples. The hypertension application may use the hypertension indicator to generate a notification to a user. The notification may include a mobile notification such as an app notification, a text notification, an email notification, or another notification that is known. The hypertension tracking application may operate using the method of FIG. 5A.

In one or more embodiments, the programs 222 may include a third-party application. The third-party application may be, for example, a third-party hypertension tracking application. The third-party application may record voice samples from the user and report the subject's hypertension indicator. Such a third-party application may communicate via an API with the prediction unit 224. Such a third-party application may integrate with a health tracker of the individual such as a Fitbit®, or Apple® Watch such that additional measurement data may be collected. The third-party application may record historical hypertension in order to determine changes in the user's hypertension indicators. The third-party application may use the hypertension indicator to generate a notification to a user. The notification may include a mobile notification such as an app notification, a text notification, an email notification, or another notification that is known. The third-party application may operate using the method of FIG. 5A.

The prediction unit 224 receives voice data from the audio source connected to I/O unit 210 via the data collection unit 226 and may transmit the voice data to the server (see e.g. 106 FIG. 1). In response, the server may operate the method as described in FIG. 5A to generate a hypertension prediction in the form of a hypertension indicator for the subject and may respond with the hypertension indicator to the user device 200. The voice sample data may be stored in the voice sample database 228 along with the hypertensions indicator. Prediction unit 224 may determine predictive messages based on the hypertension model (stored in memory) and the voice sample data. The predictive messages may be displayed to a user of the mobile device 200 using display 204. The predictive messages may include a hypertension category.

The prediction unit 224 may receive voice data and may perform the segmentation 306, the features extraction 308 based on a predetermined set of features, and generate a feature matrix 312 (e.g. FIG. 3). This may be used as input to a hypertension prediction model.

The prediction unit 224 of the mobile device 200 may include a hypertension prediction model and may operate the method as described in FIG. 5A to generate a hypertension prediction in a hypertension indicator for the subject on the mobile device itself. The hypertension prediction model of the prediction unit may include support vector classifier (SVC) model such as its extended version NuSVC, a K-nearest neighbors (KNN) model, a logistic regression (LR) model, a linear discriminant analysis (LDA) model, a quadratic discriminant analysis (QDA) model, a Gaussian naïve Bayes (GNB) model, a Bernoulli naïve Bayes (BNB) model, a Ridge model, an extreme gradient boosting (XGB) model, a decision tree (DT) model, and a random forest (RF) model. In this alternate unit, the voice sample data may be stored in the voice sample database 228 along with the hypertension indicator data.

The data collection unit 226 receives voice sample data from an audio source connected to the I/O unit 210.

The voice sample database 228 may be a database for storing voice samples received by the user device 400. The voice sample database 228 may receive the data from the data collection unit 226.

Hypertension indicator database 230 may be a database for storing generated hypertension indicators from the prediction unit 224 on the user device or the server.

Referring next to FIG. 2B, there is shown a device diagram 250 in accordance with one or more embodiments. The server device diagram 250 shows detail of the server 106 in FIG. 1. The server 250 includes one or more of a communication unit 252, a display 254, a processor unit 256, a memory unit 258, I/O unit 260, a user interface engine 264, and a power unit 266.

The communication unit 252 can include wired or wireless connection capabilities. The communication unit 252 can include a radio that communicates using standards such as IEEE 802.11a, 802.11b, 802.11g, or 802.11n. The communication unit 252 can be used by the server 250 to communicate with other devices or computers.

Communication unit 252 may communicate with a network, such as networks 104 (see FIG. 1).

The display 254 may be an LED or LCD based display and may be a touch sensitive user input device that supports gestures.

The processor unit 256 controls the operation of the server 250. The processor unit 256 can be any suitable processor, controller or digital signal processor that can provide sufficient processing power depending on the configuration, purposes and requirements of the server 250 as is known by those skilled in the art. For example, the processor unit 256 may be a high-performance general processor. In alternative embodiments, the processor unit 256 can include more than one processor with each processor being configured to perform different dedicated tasks. The processor unit 256 may include a standard processor, such as an Intel® processor or an AMD® processor.

The processor unit 256 can also execute a user interface (UI) engine 264 that is used to generate various UIs for delivery via a web application provided by the Web/API Unit 282, some examples of which are shown and described herein, such as interfaces shown in FIG. 4A-4D.

The memory unit 258 comprises software code for implementing an operating system 270, programs 272, prediction unit 274, voice sample database 278, model generation unit 280, and Web/API Unit 282.

The memory unit 258 can include RAM, ROM, one or more hard drives, one or more flash drives or some other suitable data storage elements such as disk drives, etc. The memory unit 258 is used to store an operating system 270 and programs 272 as is commonly known by those skilled in the art.

The I/O unit 260 can include at least one of a mouse, a keyboard, a touch screen, a thumbwheel, a track-pad, a track-ball, a card-reader, an audio source, a microphone, voice recognition software and the like again depending on the particular implementation of the server 250. In some cases, some of these components can be integrated with one another.

The user interface engine 264 is configured to generate interfaces for users to configure hypertension indicators, view hypertension indicators and associated notifications, view voice sample data, etc. The various interfaces generated by the user interface engine 264 may be transmitted to a user device by virtue of the Web/API Unit 282 and the communication unit 252.

The power unit 266 can be any suitable power source that provides power to the server 250 such as a power adaptor or a rechargeable battery pack depending on the implementation of the server 250 as is known by those skilled in the art.

The operating system 270 may provide various basic operational processes for the server 250. For example, the operating system 270 may be a server operating system such as Ubuntu® Linux, Microsoft® Windows Server® operating system, or another operating system.

The programs 272 include various user programs. They may include several hosted applications delivering services to users over the network, for example, hypertension tracking applications, health tracking applications, and the like.

In one or more embodiments, the programs 272 may provide a public health platform that is web-based, or client server based via a Web/API Unit 282 that provides hypertension information for a set of subjects. This may include a patient population of a medical professional who is a user of the public health platform. For example, the medical professional may be able to receive a view hypertension indicator for their patients who are subjects.

The prediction unit 274 receives voice data from a user device over a network at Web/API Unit 282 and may operate the method as described in FIG. 5A to generate a hypertension indicator for the subject. The server may respond with the hypertension indicator via a message from the Web/API Unit 282. The voice sample data may be stored in the voice sample database 278 along with the predicted hypertension indicator data. Prediction unit 274 may determine predictive messages based on the at least one predetermined voice feature and the voice sample data.

The voice sample database 278 may be a database for storing voice samples received from the one or more user devices via Web/API Unit 282. The voice sample database 278 may include voice samples from a broad population of subjects interacting with user devices. The voice samples in voice sample database 278 may be referenced by a subject identifier that corresponds to an entry in the hypertension indicator database 280. The subject identifier may be a de-identified number that provides a measure of privacy and security for the subject's information in the event that it is ever inadvertently disclosed, for example as the result of a hacking attempt. The voice sample database 278 may include voice samples for a population of subjects, including more than 10,000, more than 100,000 or more than a million subjects. The voice sample database 278 may include voice samples from many different audio sources, including passive sensor devices, user devices, smart speakers, smart watches, game systems, etc.

The hypertension indicator database 280 may be a database for storing hypertension indicator prediction by the prediction unit 274. The hypertension indicator database 280 may be referenced by a subject identifier. The subject identifier may be a de-identified number that provides a measure of privacy and security for the subject's information in the event that it is ever inadvertently disclosed, for example as the result of a hacking attempt. The hypertension indicator database 280 may include hypertension indicator database corresponding to voice samples for a population of subjects, including more than 1,000, more than 10,000 or more than 100,000 subjects.

The Web/API Unit 282 may be a web-based application or Application Programming Interface (API) such as a REST (REpresentational State Transfer) API. The API may communicate in a format such as XML, JSON, or other interchange format.

The Web/API Unit 282 may receive a hypertension indicator prediction request including a voice sample, may apply methods herein to determine a hypertension indicator prediction, and then may provide the prediction in a hypertension indicator prediction response. The voice sample, values determined from the voice sample, and other metadata about the voice sample may be stored after receipt of a hypertension indicator prediction in voice sample database 278. The predicted hypertension indicator may be associated with the voice sample database entry and stored in the hypertension indicator database 280.

The model generation unit 284 may be used in order to generate a hypertension indicator model in accordance with the methods of FIG. 5B. The hypertension indicator model may be stored in hypertension indicator database 280 and additionally may be transmitted to a user device via communication unit 252.

The model generated by model generation unit 284 may include one or more of a support vector classifier (SVC) model such as its extended version NuSVC, a K-nearest neighbors (KNN) model, a logistic regression (LR) model, a linear discriminant analysis (LDA) model, a quadratic discriminant analysis (QDA) model, a Gaussian naïve Bayes (GNB) model, a Bernoulli naïve Bayes (BNB) model, a Ridge model, an extreme gradient boosting (XGB) model, a decision tree (DT) model, and a random forest (RF) model.

Referring next to FIG. 3, there is shown a model development diagram 300 in accordance with one or more embodiments. The model development diagram 300 in conjunction with FIG. 5B describes the data collection and model generation method for the hypertension indicator models described herein.

At 302, voice sample data was collected from a population of subjects. At the time of enrollment in the example, participant age, height, weight, BMI, heart rate, and blood pressure were measured and recorded, and heart rate and blood pressure were recorded again at the end of the data collection period.

Participants may be excluded if they received a Type 2 diabetes or prediabetes diagnosis by a physician, as recent research indicates that there are vocal changes associated with Type 2 diabetes diagnosis.

During the example data collection period 302, subjects voice samples were collected. The subjects may be instructed, for example, to record the speech segment “Hello, how are you? What is my glucose level right now?” up to six times daily for two weeks into a custom mobile application. Voice recordings may be stored using a sampling frequency of 44.1 KHz and then uploaded from the app to a secure web server (for example, server 106 in FIG. 1). Participants may be anonymized and may be assigned an alphanumeric participant ID or subject identifier.

The participants may be annotated by a clinician as “hypertensive” based on the average of SBP and DBP measurements at the beginning of the example and after the final speech recording task. The thresholds used in this example are 1) SBP≥135 OR DBP≥85, which is selected to balance the dataset regarding the number of hypertensive and non-hypertensive samples and derived from the arithmetic average of the AHA and ESH guidelines for hypertension, and II) SBP≥140 OR DBP≥90, which is selected as per ESH hypertension guideline.

At 304, all of the voice recordings from the subject population were input to a model training algorithm.

At 306, each recording may be segmented based on one of three frameworks. These may include whole recording where features may be extracted from the entire speech recording, quarter-based where each recording may be divided into four equidistant quarters and features may be extracted from each quarter, and stacked-quarter where features from all four quarters may be concatenated to form a comprehensive feature matrix. The quarter segments may include four equidistant quarters of each voice sample.

At 308 features were extracted from all of the whole voice samples, and for each quarter segment generated at 306. These may include the features identified in FIG. 6.

The features may be grouped into four categories: non-linear features, low-level descriptor features, time-related features, and frequency-related features.

The non-linear features may include Shannon and logarithm entropies.

The low-level descriptor features may include the first four statistical moments (mean, variance, kurtosis, and skewness), the range (difference between peak and valley of the recording), and the cumulative sum of the first derivative (CSFD) of the signal, defined as Equation (3) in Example 2 below, where x[n] is the input speech recording of length N.

The time-related features may include the mean and standard deviation of the zero-crossing rate over different frames of the input signal.

The frequency-related features may include the mean and standard deviation of 13 Mel-frequency cepstral coefficients (MFCCs), ΔMFCCs, and ΔΔMFCCs, mean and standard deviation of 12 chromagram coefficients, mean and standard deviation of spectral contrast (7 coefficients), centroid, bandwidth, flatness, and roll-off, the fundamental frequency, LLDs of the tempogram, six equidistant spanned power spectral density (PSD) from 0 to

$$\frac{{F}_{s}}{2} Hz ,$$

and LLDs and entropies of PSD, as well as the third coefficients of the polynomial curve fitted to the PSD.

The feature extraction 308 may use the quarter segments in a cross-quarter framework, where the features from two subsequent quarters may be subtracted and the resultant features are used for model development.

The feature 308 the spectral features from the frequency-related subset may be extracted using a Hanning window, for example with a size of 2048 samples and a hop size of 512 samples. The PSD may be generated using the Welch method, for example with a Hanning window size of 100 ms and a 75% overlap. The frequency resolution of the PSD may be four times greater than the window size.

At 310 features may be selected. Since the number of features in both quarter-based or stack-based models may be high, the utilization of feature selection algorithms may be important. In this regard, the Least Absolute Shrinkage and Selection Operator (LASSO) method may be used to select features. The LASSO method may use the objective function in Equation (1) below, where N is the number of samples, X is the feature matrix, β is the coefficient vector corresponding to each feature column of X, y is the original label vector, and α is the hyper-parameter that shows the trade-off between penalizing the coefficient and the regression parts of the objective function.

a may be set to a standard value of 0.01. Afterwards, the features that have non-zero coefficients are selected for the model development stage. The number of selected features is annotated with Nₛ herein.

At 310, the dimensionality of the features may be reduced. This may include processing the features, for example using Searching for Uncorrelated List of Variables (SULOV) and Least Absolute Shrinkage and Selection Operator (LASSO). First, SULOV may be applied to remove features highly correlated with other features. For example, the SULOV may be performed with a correlation threshold set at 0.8. Next, LASSO may be performed to penalize less contributory features in a linear regression model, for example with the a parameter set at 0.01.

For selecting the features, 80% of the data, randomly shuffled in each iteration, may be used to run the feature selection models. This procedure may be repeated for 5 iterations. Features that appeared in at least three iterations may be selected for the final model development stage. The number of selected features may be denoted as Nₛ herein.

At 312 a feature matrix is constructed by applying step 310 for all participants or subjects. This may include concatenating the features from all four quarters to create a large feature matrix in the stacked quarters framework.

At 314, the average for each feature over multiple recordings may be taken and considered as the feature for the participant.

At 316, feature standardization may be performed including zero-mean and unit variance determinations.

At 318, the Leave-One-Subject-Out (LOSO) analysis may be conducted. This includes SMOTE 320, classification 322 and hypertension indicator model generation 324.

The LOSO approach may be used to evaluate the training. LOSO works by leaving out the feature vector of one subject and training the model is based on the remaining subjects, then the procedure is repeated until all subjects are tested and their corresponding label is predicted. To report the performance of different models, the balanced accuracy (BACC) may be reported which includes both sensitivity and specificity and is defined as the average of true positive rate (TPR) and true negative rate (TNR), which are calculated based on Equation (2) below, where TP, TN, FP, FN, represented true positive (correctly predicted as hypertensive), true negative (correctly predicted as non-hypertensive), false positive (incorrectly predicted as hypertensive), and false negative (incorrectly predicted as non-hypertensive), respectively.

At 320, a synthetic minority oversampling technique (SMOTE) may be employed to increase the number of samples in the hypertensive class. In the SMOTE algorithm, first, a sample in the minority class is selected (xᵢ). Afterwards, another sample from it's k nearest neighbors is selected (x_{i←k}). Using linear interpolation, a new sample (\tilde{x}ᵢ) is generated through (\tilde{x}ᵢ=xᵢ+ [symbol] (0,1)(x_{i←k}−xᵢ) where [symbol] (0,1) is a random uniform number between 0 and 1. This procedure is repeated until the number of samples in both hypertensive and non-hypertensive are almost the same. In this example, we have used k=7, unless explicitly specified otherwise, and the SMOTE technique was applied only on the training portion of the data and for the sake of enhancing the generalizability of the developed model.

At 322, a group of 12 classical machine learning models, namely support vector classifier (SVC) and its extended version NuSVC, K-nearest neighbours (KNN), logistic regression (LR), linear discriminant analysis (LDA), quadratic discriminant analysis (QDA), Gaussian naïve Bayesian (GNB), Bernoulli naïve Bayesian (BNB), Ridge, extra gradient boosting (XGBoost), decision tree (DT), and random forest (RF) are used for the model development. More details about the classifiers' hyperparameters can be found in FIG. 7. Since the speech characteristics of males and females are different and building a single model for both sexes would likely hurt the performance of the model, we have developed a model for men and women separately. Furthermore, despite trying several classifiers, only those with the best performance are reported in the results.

The models may be evaluated using a leave-one-subject-out (LOSO) approach. In this approach, the feature vector of each participant may be left out as the test set, while the remaining feature vectors form the training feature matrix. This procedure may be repeated for all participants. To measure the performance of the different models, balanced accuracy (BACC) may be used, which is the arithmetic average of sensitivity and specificity, and the area under the receiver operating characteristic curve (AuROC).

At 324, a hypertension indicator model is generated.

Referring next to FIGS. 4A, 4B, 4C, and 4D together, there are example user interfaces 400, 410, 420, 430, respectively showing a subject collecting a voice sample and receiving a hypertension indicator.

At the first execution of the application shown in FIGS. 4A, 4B, 4C, and 4D, the user may be required to enter onboarding information to configure the application. The onboarding may be performed the first time the user opens the application, or subsequently as requested by the user.

The onboarding information can include participant age, height, weight, BMI, heart rate, and blood pressure were measured and recorded, and heart rate and blood pressure.

At interface 400, there is a user interface shown to a user at a user device 402 who desires to receive a hypertension indicator. To initiate the prediction, the user is prompted to begin the hypertension check by selecting a start button 406. Once start is selected, the audio input of the user device begins recording the voice sample into memory of the user device 402.

In an alternate embodiment, the user may receive a notification on the user device 402 to initiate the voice sampling, and by selecting the notification may be presented with interface 400 to initiate the collection. The notification to the user to initiate the voice sampling may be determined based on the time of day.

In response to the user selecting the start button, a variable prompt interface 410 is shown, prompting the user to read the prompt 414. The prompt may be a variable prompt 414 as shown, and may change subject to subject, or for each voice sample that is recorded. During the voice sample collection, the user interface 410 may show a voice sample waveform 416 on the display.

Alternatively, a static prompt to user interface 420 may instead be shown to a subject and the prompt 424 may be static. Each subject may speak the same prompt out loud for every voice sample. During the voice sample collection, the user interface 420 may show a voice sample waveform 426 on the display.

In the case of either the interface 410 or the interface 420, the interface may further ask the user one or more questions at the time the user reads the prompt 414 or 424. For example, the interface may ask the user “Have you measured your blood pressure today” and may record a text-based answer. Alternatively, a voice prompt may follow the user reading the prompt 414 or 424.

In response to completing the voice prompt (either static or variable), a hypertension indicator may be made in a hypertension indicator prediction interface 430. The hypertension prediction or hypertension indicator 434 may be a categorical prediction, i.e. ‘Low’, ‘Medium’, ‘High’, and ‘Very High”. The hypertension indicator 434 may be a numerical prediction (see e.g. chance of hypertension) including a percentage. As described herein, the hypertension indicator 434 may be for a plurality of predictions including two or more of the above noted categorical, indicator of days, or numerical prediction. The prediction may be generated by a server or may be generated by the user device itself.

Referring next to FIG. 5A, there is shown a computer-implemented method diagram 500 in accordance with one or more embodiments. The method 500 is a computer-implemented method for providing a hypertension indicator for a subject.

At 502, a hypertension indicator prediction model is provided at a memory.

At 504, a processor in communication with the memory receives a voice sample from the subject.

At 506, at least one voice biomarker feature value from the voice sample is extracted at the processor for at least one predetermined voice biomarker feature. For example, the feature extraction may be as described at 308 (e.g. FIG. 3). The features extracted may include the features identified in FIG. 6.

The features may be grouped into four categories: non-linear features, low-level descriptor features, time-related features, and frequency-related features.

The non-linear features may include Shannon and logarithm entropies.

The low-level descriptor features may include the first four statistical moments (mean, variance, kurtosis, and skewness), the range (difference between peak and valley of the recording), and the cumulative sum of the first derivative (CSFD) of the signal, defined as Equation (3) in Example 2 below, where x[n] is the input speech recording of length N.

The time-related features may include the mean and standard deviation of the zero-crossing rate over different frames of the input signal.

The frequency-related features may include the mean and standard deviation of 13 Mel-frequency cepstral coefficients (MFCCs), ΔMFCCs, and ΔΔMFCCs, mean and standard deviation of 12 chromagram coefficients, mean and standard deviation of spectral contrast (7 coefficients), centroid, bandwidth, flatness, and roll-off, the fundamental frequency, LLDs of the tempogram, six equidistant spanned power spectral density (PSD) from 0 to

$$\frac{{F}_{s}}{2} Hz ,$$

and LLDs and entropies of PSD, as well as the third coefficients of the polynomial curve fitted to the PSD.

At 508, the hypertension indicator for the subject is determined at the processor based on the at least one voice biomarker feature value and the hypertension indicator prediction model.

At 510, the hypertension indicator for the subject or an output based on the hypertension indicator is output at an output device.

In one or more embodiments, the hypertension indicator for the subject may be one of a plurality of categories.

In one or more embodiments, the hypertension indicator may be a category based on European Society of Hypertension (ESH) hypertension guidelines.

In one or more embodiments, the plurality of categories may comprise: a first hypertensive category comprising SBP>=135 OR DBP>=85; and a non-hypertensive category.

In one or more embodiments, the plurality of categories may comprise: a second hypertensive category comprising SBP>=140 OR DBP>=90.

In one or more embodiments, the at least one predetermined voice biomarker feature may be selected from the group of a non-linear feature, a low-level descriptor feature, a time related feature, and a frequency-related feature.

In one or more embodiments, the non-linear feature may comprise one of a Shannon entropy of the voice sample and a logarithm entropy of the voice sample, the low-level descriptor feature may comprise one of a mean of the voice sample, a variance of the voice sample, a skewness of the voice sample, a kurtosis of the voice sample, a range of the voice sample, a cumulative sum of the first derivative (CSFD) of the signal, the time related feature may comprise one of a mean of the zero-crossing rate, a standard deviation of the zero-crossing rate, and the frequency-related feature may comprise one of a mean Mel-scale frequency cepstral coefficient (MFCC), a standard deviation of a Mel-scale frequency cepstral coefficient (MFCC), a first (ΔMFCC) and a second derivative (ΔΔMFCC) of the MFCC, a mean Chromagram coefficient, a standard deviation of a Chromagram coefficient, a mean spectral contrast, a standard deviation of spectral contrast, a centroid of a spectral contrast, a bandwidth of a spectral contrast, a flatness of a spectral contrast, a roll-off of a spectral contrast, a fundamental frequency, an LLD of a tempogram, a power spectral density (PSD) from 0 to

$$\frac{{F}_{s}}{2} Hz ,$$

an LLD of the PSD, an entropies of the PSD, and a coefficient of a polynomial curve fitted to the PSD.

In one or more embodiments, the hypertension indicator prediction model may comprise a statistical classifier.

In one or more embodiments, the statistical classifier may comprise at least one selected from the group of: a support vector classifier (SVC), a K-nearest neighbour (KNN) classifier, a logistic regression (LR) classifier, a linear discriminant analysis (LDA) classifier, quadratic discriminant analysis (QDA) classifier, a Gaussian naïve Bayesian (GNB) classifier, Bernoulli naïve Bayesian (BNB) classifier, Ridge, an extra gradient boosting (XGBoost) classifier, decision tree (DT) classifier, and random forest (RF) classifier.

In one or more embodiments, the hypertension indicator prediction model may be an ensemble model, the ensemble model comprising n LDA classifiers; and wherein the determining, at the processor, the hypertension indicator may comprise: determining a prediction from each of the n LDA classifiers in the ensemble model; and determining the hypertension indicator based on an election of the predictions from the n LDA classifiers in the ensemble model.

In one or more embodiments, the method may further comprise: transmitting, to a user device in network communication with the processor, the hypertension indicator for the subject, wherein the outputting of the hypertension indicator for the subject occurs at the user device.

In one or more embodiments, the method may further comprise determining the hypertension indicator for the subject based on at least one clinicopathological value for the subject, optionally at least one of height, weight, BMI, diabetes status and blood pressure.

In one or more embodiments, the voice sample may comprise a predetermined phrase vocalized by the subject.

In one or more embodiments, the voice sample may comprise a randomly selected phrase vocalized by the at least one subject.

In one or more embodiments, the predetermined phrase or the randomly selected phrase may be displayed to the subject on the user device.

In one or more embodiments, the voice sample may be received from an audio sensor at the user device.

Referring next to FIG. 5B, there is shown another computer-implemented method diagram 550 in accordance with one or more embodiments. The method 550 is a computer-implemented method for generating a hypertension indicator prediction model.

At 552, providing, at a memory: a plurality of voice samples from at least one subject at a plurality of time points; and a plurality of blood pressure measurements, wherein each blood pressure measurement comprises a diastolic measurement and a systolic measurement, and each blood pressure measurement is associated with a corresponding subject in the at least one subject. The plurality of voice samples from at least one subject may be associated with a hypertensive category label. For example, a first hypertensive category used to label each voice sample may be SBP≥140 mmHg OR DBP≥90 mmHg, selected based on European Society of Hypertension (ESH) hypertension guideline. A second hypertensive category that may be used to label each voice sample may be SBP≥135 mmHg OR DBP≥85 mmHg.

At 554, the plurality of voice samples are sorted at a processor in communication with the memory into two or more hypertensive categories based on the blood pressure measurement.

At 556, voice feature values for a set of voice features from each of the plurality of voice samples are extracted at the processor.

At 558 a subset of voice features from the set of voice features are selected at the processor.

At 560, the hypertension indicator prediction model is generated at the processor based on the subset of voice features.

In one or more embodiments, generating the hypertension indicator prediction model based on the subset of voice features may comprise determining a weight for each voice feature in the subset of voice features.

In one or more embodiments, the method may further comprise: selecting, at the processor, a subset of voice features from the set of voice features based on a Least Absolute Shrinkage and Selection Operator (LASSO) method.

In one or more embodiments, the LASSO objective function may be is

$${min}_{β} \frac{1}{2 N} {‖ X β - y ‖}_{2}^{2} + α {‖ β ‖}_{1} .$$

In one or more embodiments, each of the voice features may be selected from the group of a non-linear feature, a low-level descriptor feature, a time related feature, and a frequency-related feature.

In one or more embodiments, the non-linear feature may comprise one of a Shannon entropy of the voice sample and a logarithm entropy of the voice sample, the low-level descriptor feature may comprise one of a mean of the voice sample, a variance of the voice sample, a skewness of the voice sample, a kurtosis of the voice sample, a range of the voice sample, a cumulative sum of the first derivative (CSFD) of the signal, the time related feature may comprise one of a mean of the zero-crossing rate, a standard deviation of the zero-crossing rate, and the frequency-related feature may comprise one of a mean Mel-scale frequency cepstral coefficient (MFCC), a standard deviation of a Mel-scale frequency cepstral coefficient (MFCC), a first (ΔMFCC) and a second derivative (ΔΔMFCC) of the MFCC, a mean Chromagram coefficient, a standard deviation of a Chromagram coefficient, a mean spectral contrast, a standard deviation of spectral contrast, a centroid of a spectral contrast, a bandwidth of a spectral contrast, a flatness of a spectral contrast, a roll-off of a spectral contrast, a fundamental frequency, an LLD of a tempogram, a power spectral density (PSD) from 0 to

$$\frac{{F}_{s}}{2} Hz ,$$

an LLD of the PSD, an entropies of the PSD, and a coefficient of a polynomial curve fitted to the PSD.

In one or more embodiments, the method may further comprise: dividing, at the processor, each of the plurality of voice samples a plurality of equidistant segments; extracting, at the processor, voice feature values for a set of voice features from each of the plurality of equidistant segments; and wherein the voice feature values for each voice sample may comprise a concatenation of the voice feature values for the corresponding plurality of equidistant segments.

In one or more embodiments, the method may further comprise: averaging, at the processor, at least two voice samples in the plurality of voice samples, the at least two voice samples corresponding to the same subject.

In one or more embodiments, the method may further comprise: applying, at the processor, a synthetic minority oversampling technique (SMOTE) to increase a number of the plurality of voice samples.

In one or more embodiments, the method may further comprise: determining, at the processor, a true positive rate (TPR) and a true negative rate (TNR) for the generated hypertension indicator prediction model using a leave-one-subject-out (LOSO) method.

In one or more embodiments, the two or more hypertensive categories may comprise at least a first hypertensive category comprising SBP>=135 OR DBP>=85 and a second hypertensive category comprising SBP>=140 OR DBP>=90.

In one or more embodiments, the voice sample may comprise a predetermined phrase vocalized by the at least one subject.

In one or more embodiments, the voice sample may comprise a randomly selected phrase vocalized by the at least one subject.

While the above description provides examples of one or more processes or systems, or computer program products, it will be appreciated that other processes or systems, or computer program products may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

### EXAMPLES

### Example 1: Machine Learning-Enabled Hypertension Detection Through Acoustical Speech Analysis: Model Development and Validation

An example was performed to investigate a framework for detecting hypertension through acoustic analysis of speech was evaluated. By recording speech across multiple sessions and analyzing its temporal and spectral characteristics, indicators of hypertension were identified. Two thresholds for labeling individuals with hypertension were used: (I) systolic blood pressure (SBP)≥135 mmHg or diastolic blood pressure (DBP)≥85 mmHg, and (II) SBP≥140 mmHg or DBP≥90 mmHg. Example #1 involved 245 participants, including 91 females. Predictive models were developed for each gender and their performance was assessed using leave-one-subject-out validation. For the first threshold, the balanced accuracy achieved was 84% for females and 77% for males. For the second threshold, the corresponding balanced accuracies were 63% for females and 86% for males. These results demonstrate the potential of utilizing speech-based representations for non-invasive screening of hypertension.

The example analyzed acoustic speech features from recordings of 235 participants, classifying them according to two criteria (SBP≥135 mmHg or DBP≥85 mmHg, and SBP≥140 mmHg or DBP≥90 mmHg). Feature selection techniques were implemented and integrated 12 different classifiers for each gender to enhance the robustness and accuracy of the analyses. Moreover, a leave-one-subject-out validation method was employed to report our findings, ensuring a thorough and individualized assessment of the model's performance across all participants.

### Methods

### Dataset

Participants were recruited as part of a larger example assessing the effects of glucose levels on the voice (Clinical Trial Identifier: (CTRI/2021/08/035957). Participant age, height, weight, BMI, heart rate, and blood pressure were measured and recorded at _{recruitment}, and heart rate and blood pressure were recorded again at the end of the data collection period. All participants were non-smokers. All participants signed informed consent. Participants were excluded if they received a Type 2 diabetes or prediabetes diagnosis by a physician, as recent research indicates that there are vocal changes associated with Type 2 diabetes diagnosis (19).

Participants were instructed to record the speech segment “Hello, how are you? What is my glucose level right now?” up to six times daily for two weeks into a custom mobile application. Voice recordings were stored using a sampling frequency of 44.1 KHz and then uploaded from the app to a secure web server, where they could only be accessed with a private API key by our research team. All participants were anonymized and were assigned an alphanumeric participant ID. No identifying information was collected in the voice recordings.

The participants were annotated as ‘hypertensive” based on the average of SBP and DBP measurements in the beginning of the example and after the final speech recording task. The thresholds used in this example are 1) SBP≥135 OR DBP≥85 which is selected to balance the dataset regarding the number of hypertensive and non-hypertensive samples and derived from the arithmetic average of the AHA and ESH guidelines or hypertension (20), (21) and II) SBP≥140 OR DBP≥90 which is selected as per ESH hypertension guideline (20).

### Feature Extraction

The feature set covered a wide range of characteristics including temporal, spectral, and spectrotemporal features. Temporal features include low-level descriptors (e.g., mean, variance, skewness, kurtosis, and range) of the input signal, mean and standard deviation of zero-crossing rate over time, nonlinear features of the input signal such as Shannon and logarithm entropies. Spectral characteristics include mean and standard deviation of spectral contrast, centroid, bandwidth, flatness, and roll-off over time, fundamental frequency (F0), low-level descriptors of tempogram, bandpowers, mean, root-mean-square, skewness, range, entropies, cumulative different sum (CDS) and mean and standard deviation of the 3rd-level polynomial fitting to the curve of power spectral density (PSD). The spectrotemporal features encompass mean and standard deviation of 13 Mel-scale frequency cepstral coefficients (MFCCs), its first (ΔMFCC) and second derivatives (ΔΔMFCC) over the time and mean and standard deviation of 12 Chromagram coefficients over the time. The spectral features are extracted through framing each recording using a Hanning window of size of 2048 samples and hop size of 512 samples. The PSD is also generated through Welch method with Hanning widow of size of 4410 samples (equivalent to 100 ms) and 75% overlap, and of frequency size of 4 times greater than the window size. The full list of features is shown in FIG. 6.

As a result of this, 160 features are extracted for each signal. In this example, we investigated the possibility of using speech features in four different frameworks:
We extracted features from the whole speech recording.
In the next stage, we divided each recording into four equidistant quarters and extracted features from each quarter of the recording, called the quarter-based framework.
We also explored the speech dynamics through the cross-quarter framework, where the features from two subsequent quarters are subtracted and the resultant features are used for model development.
Finally, we concatenated all the features from all four quarters to create a huge feature matrix (640 features), in the stacked quarters framework.

Once the features are extracted for all recordings, the average of features is computed per subject. Therefore, each participant has a unique feature vector with a length of either 160 or 640, depending on the framework, which will be used in the next stages of the model development.

### Feature Selection

Since the number of features in both quarter-based or stack-based models are high, the utilization of feature selection algorithms is crucial. In this regard, we used the Least Absolute Shrinkage and Selection Operator (LASSO) method, through which the following objective function is optimized:

$${min}_{β} \frac{1}{2 N} {‖ X β - y ‖}_{2}^{2} + α {‖ β ‖}_{1} \qquad Equation ( 1 )$$

where N is the number of samples, X is the feature matrix, β is the coefficient vector corresponding to each feature column of X, y is the original label vector, and α is the hyperparameter that shows the trade-off between penalizing the coefficient and the regression parts of the objective function. For all frameworks developed in this example, we have set a to a standard value of 0.01, unless explicitly specified otherwise. Afterwards, the features that have non-zero coefficients are selected for the model development stage. The number of selected features is annotated with Nₛ herein.

### Model Development

As a result of the lower number of hypertensive cases in our dataset, there was a surge to tackle the imbalance issue. In this regard, we applied the synthetic minority oversampling technique (SMOTE) to increase the number of samples in the hypertensive class (22). In the SMOTE algorithm, first, a sample in the minority class is selected (xᵢ). Afterwards, another sample from it's k nearest neighbors is selected (x_{i←k}). Using linear interpolation, a new sample (\tilde{x}ᵢ) is generated through (\tilde{x}ᵢ=xᵢ+ [symbol] (0,1)(x_{i←k}−xᵢ) where [symbol] (0,1) is a random uniform number between 0 and 1. This procedure is repeated until the number of samples in both hypertensive and non-hypertensive are almost the same. In this example, we have used k=7, unless explicitly specified otherwise, and the SMOTE technique was applied only on the training portion of the data and for the sake of enhancing the generalizability of the developed model.

In the next step, a group of 12 classical machine learning models, namely support vector classifier (SVC) and its extended version NuSVC, K-nearest neighbours (KNN), logistic regression (LR), linear discriminant analysis (LDA), quadratic discriminant analysis (QDA), Gaussian naïve Bayesian (GNB), Bernoulli naïve Bayesian (BNB), Ridge, extra gradient boosting (XGBoost), decision tree (DT), and random forest (RF) are used for the model development. More details about the classifiers' hyperparameters can be found in FIG. 7. Since the speech characteristics of males and females are different and building a single model for both sexes would likely hurt the performance of the model, we have developed a model for men and women separately. Furthermore, despite trying several classifiers, only those with the best performance are reported in the results.

### Evaluation

In other to fairly evaluate the developed frameworks, we followed the leave-one-subject-out (LOSO) approach, through which the feature vector of one subject is left out and the model is trained based on the remaining subject, then the procedure is repeated until all subjects are tested and their corresponding label is predicted. To report the performance of different models, we would report balanced accuracy (BACC) which includes both sensitivity and specificity and is defined as the average of true positive rate (TPR) and true negative rate (TNR), which are calculated as

$$TPR = \frac{TP}{TP + {FN}^{′}} \qquad Equation ( 2 )$$

$$TNR = \frac{TN}{TN + {FP}^{′}}$$

where TP, TN, FP, FN, represented true positive (correctly predicted as hypertensive), true negative (correctly predicted as non-hypertensive), false positive (incorrectly predicted as hypertensive), and false negative (incorrectly predicted as non-hypertensive), respectively. The process of model development is provided in further detail in FIG. 14.

### Software Used for Analyses

All the analyses have been conducted in Python 3.11.7. Speech-related features are extracted using Numpy (version 1.26.3) with the help of Librosa (0.10.1) (23). All machine learning models, and feature selection techniques are implemented using the Scikit-learn package (version 1.2.2) (24). FIG. 7 refers to the details of the classifiers used in the proposed method.

### Extracted Features

Referring to FIG. 6 there is provided a list of the extracted features. s[n], Pₛ, and Ps[f1, f2] are N-sample signal s, power spectrum of s[n], and normalized band power of s[n] in the frequency band between f1 and f2 (in Hz), which can be denoted as

$${\int }_{{f}_{1}}^{{f}_{2}} {P}_{s} df / {\int }_{0}^{{f}_{s}} {P}_{s} df ’$$

respectively. The CDS is also defined as

$${\sum }_{n = 1}^{N} s [ n ] - s [ n - 1 ] .$$

μt and σt are mean and standard deviation operators, respectively, that are applied through the output time frames of the target feature. TP, TN, FP, FN, represented true positive (correctly predicted as hypertensive), true negative (correctly predicted as non-hypertensive), false positive (incorrectly predicted as hypertensive), and false negative (incorrectly predicted as non hypertensive), respectively.

### Results

### Participant Demographics

In total, 245 participants completed the research protocol. More details about the participants' demographics based on different thresholds are reflected in FIGS. 8 and 9.

### Whole Recording

The result of the first framework which involves extracting features from the whole speech recording is shown in FIG. 10. As indicated by the table, the predictive model performs better for females under the more relaxed threshold, whereas the opposite is observed for males. Additionally, the number of selected features for the second threshold is approximately half of that for the first threshold, suggesting lower complexity in the models of the second threshold.

### Quarter-Based Performance

The classification results for the proposed quarter-based pipeline in both hypertension criteria are presented in FIG. 11. The table reveals that the most optimal performance was attained using features extracted from the second quarter of the recordings in both criteria. This suggests that the second quarter contains words, phrases, or vowels that harbour more discriminative information about hypertension.

Additionally, the table highlights that the reported BACC in models developed for females consistently exceeds 70% in both criteria, with an impressive BACC of 79% achieved in the first criterion. However, as the criteria become more stringent, there is a notable decline in the number of hypertensive females (from 23 to 10), potentially resulting in a 7% decrease in network performance. Conversely, in males, a different trend is observed, indicating that the threshold used for the first criterion involved men exhibiting fewer symptomatic hypertension indications in the hypertensive group, and accordingly resulted in a less accurate model for hypertension identification in males.

### Cross-Quarter Performance

The results of the second pipeline, designed to capture the dynamics of acoustic features across subsequent quarters and their impact on predictive models, are presented in FIG. 12. The findings indicate that the differential features across the second and third quarters showcase the best performance in both hypertension criteria. This infers that the third quarter contains hypertension-informative aspects within the speech recordings.

Moreover, upon comparing FIGS. 11 and 12, it can be inferred that the performance of the cross-quarter pipeline is inferior to that of the single-quarter approach in females, yet superior in males. This observation implies that the discriminatory features are more prevalent across stationary segments rather than the dynamic variances within subsequent periods of recordings, particularly among females compared to males. Furthermore, the total number of selected features within the cross-quarter framework exceeds that of the single quarter-based models, indicating greater complexity in developing models using the former approach.

### Stacked Quarters

Integrating features from all quarters, as depicted in FIG. 13, significantly enhanced the performance of the models. However, given the substantial number of features and a higher number of samples for males, the number of selected features remains relatively high (166 and 149) in both criteria. This indicates that the complexity of the model necessitated the inclusion of a significant number of features.

Another notable observation is that the best-performing models are based on LDA, which exhibits markedly simpler characteristics compared to other classifiers. Consistent with findings presented in FIGS. 11 and 12, applying stricter blood pressure thresholds for hypertension case annotation led to the exclusion of marginally hypertensive male samples, which in turn improved prediction performance.

### The Effect of Recording Numbers

We demonstrated that our proposed model, when using all available recordings for each participant in a stacked quarter scenario, achieves acceptable performance in classifying hypertensive individuals. To further explore the impact of the number of recordings on evaluation outcomes, we conducted an ablation example. In this example, we used varying numbers of recordings from the set of {1, 2, 3, 4, 5, 10, 20, 30, 40, 50}, to create the test feature vectors within the LOSO validation scheme. If fewer recordings than the specified number are available for a participant, all available recordings are used.

The results, depicted in FIGS. 14A and 14B, illustrate that using a single recording significantly underperforms compared to scenarios using all available recordings.

In general, except in the model for females using a threshold of SBP≥140 OR DBP≥90, BACC trends upward as the number of recordings increases, which could stem from the fact that more recordings, gathered at various times between initial and final blood pressure measurements, provide a more robust representation of the participant's hypertension status.

In other words, incorporating more recordings improves the model's generalizability by reducing the risk of relying on a single-instance state of the participant.

### Discussion

In this example, we introduced different frameworks for screening hypertension based on speech recordings. We extended the depth of the findings by investigating different thresholds for annotating the individuals as “hypertensive” or “non-hypertensive”. Furthermore, to capture the effect of speech dynamics on the performance of the models, we split each recording into four quarters and repeated the analysis based on the features from each quarter, difference of the subsequent quarters, and the stacked feature set from all quarters.

In prior research, several studies have focused on identifying symptoms of heart failure through speech analysis (12-15). The primary observation leading to these model developments is the interconnected relationship between edema levels, exclusive to congestive/pulmonary heart failure, and vocal fold dynamics, which directly impact speech (14). Furthermore, Sara et al. (25) extracted Mel-frequency cepstral coefficients (MFCCs), pitch and formant measures, jitter, shimmer, and loudness, revealing a significant association between these features and pulmonary hypertension. However, no studies have directly investigated the physiological link between speech and arterial blood pressure.

Some studies have explored the effect of various speech conditions on blood pressure. For instance, Alvear et al. (26) demonstrated a significant association between fundamental voice frequency during mental tasks and exposure to cold temperatures with heart rate and blood pressure. Lynch et al. (27) showed that the initiation of human speech itself results in changes in SBP, DBP, and heart rate. On the other hand, various mediating factors affect both vocal cords and blood pressure, such as smoking (28,29), stress (30,31), obesity (32,33), thyroid disorders (34,35), diabetes (36,37), and sleep apnea (38,39). Therefore, while hypertension may not directly impact voice, it can influence it through these mediating factors.

In the literature on studies attempting to predict hypertension from speech recordings, only Ankicshan (18) provided a comprehensive approach by integrating vowel articulation (/a/) recordings and extracting 630-dimension acoustic features from 86 participants (30 females), achieving an accuracy of 93% and a root-mean-square-error (RMSE) of blood pressure estimation of 0.236. Although the reported metrics are high and offer further insights for other studies in the field, the validation scheme employed was K-fold cross-validation. This validation approach fails to ensure that data from the same subject are confined to a single fold, thereby increasing the risk of data leakage and biasing the results. Therefore, the generalizability of their proposed approach is questionable. This suggested us to use of the LOSO approach in reporting example results and comparing the performance of developed predictive models.

Our results suggest that segmenting the recordings into four equidistant quarters could enhance the resolution of the analysis, particularly in identifying the most hypertension-related segment. Additionally, stacking the features from all quarters helped incorporate finer discriminative details across the recording. The best-performing model for females was achieved using the threshold of SBP≥135 OR DBP≥85, employing 64 features and an LDA classifier. Conversely, the best model for males was achieved using a stricter threshold and a much larger number of features (149). Despite the large number of features, the utilization of the LOSO approach mitigates the curse of dimensionality, thereby validating the reported results. Moreover, the inconsistent behaviour between the performance of the models across different thresholds may be attributed to the low number of participants in the hypertensive class for both thresholds. Furthermore, the high number of selected features for male models suggests that the distribution of hypertension cases among males is highly sensitive to the inclusion of a new sample in the male group.

### Conclusion

This example aimed to introduce predictive models for hypertension screening using speech recordings. Various approaches based on spectrotemporal acoustic features were explored, including a squeezing technique to represent each participant with a single feature vector. Employing several classical classifiers and developing separate predictive models for each sex, we achieved notable balanced accuracy rates of 84% and 77% for females and males, respectively, using the smoother threshold of SBP≥135 OR DBP≥85. For the stricter threshold, the achieved rates were 63% and 86% for females and males, respectively, utilizing the LOSO approach. These insights pave the way for further development of generalizable, non-invasive, accessible, and cuffless hypertension screening methods utilizing speech analysis.

### Example 2: Hypertension Screening Using Acoustic Analysis and Machine Learning of Random Speech Samples: A Feasibility Study

A further example was performed on a separate cohort from Example 1. The example design and methods were similar to those described in Example 1, except as noted below. In Example 2, acoustic-based hypertension screening models were developed using covert speech from 573 participants (197 women). The speech corpora, consisting of words and short sentences, were different for each participant, as they were asked to read randomly selected sentences during multiple recordings over the course of two weeks.

### Introduction

Hypertension is the most common risk factor for a wide variety of cardiovascular conditions, such as heart failure and stroke, leading to high rates of morbidity and mortality worldwide (48). It is estimated that over one and a half billion people worldwide will be affected by hypertension by 2025 (48). A recent global survey of over 1.5 million adults reported that among those receiving treatment for high blood pressure (BP), more than 71% had uncontrolled BP (>130/80 mmHg) (49, 50). While population growth, increasing life expectancy, unhealthy lifestyles, and an aging population have contributed to the rise in the global burden of hypertension over the past decades, the actual burden is likely even higher, as more than one-third of people worldwide with hypertension remain undiagnosed (47, 51).

One way to reduce the burden of hypertension is through early detection and treatment (52). Ambulatory blood pressure measurement using an inflatable cuff, which is wrapped around the arm and estimates arterial pressure in terms of systolic pressure (SBP) and diastolic pressure (DBP) by listening to Korotkoff sounds, has been widely used as a standard diagnostic method. Although this method is effective, accurate, and non-invasive, it remains inaccessible to many people living in rural or remote areas.

Speech, as an accessible and easy-to-collect medium capable of demonstrating vocal characteristics, has been widely used in previous research for estimating and screening heart failure (53, 54, 55, 56). Additionally, it has been shown that speech and voice can reflect the degree of various conditions such as diabetes (57, 58), sleep apnea (59), and Alzheimer's disease (60). Therefore, investigating the feasibility of developing speech-based hypertension diagnostics is worthwhile.

Previously, only one study by Ankişhan explored the mentioned association and developed an acoustic model for hypertension screening (61). Their study was based on 630 acoustic features extracted from 86 participants (40 women) articulating the vowel /a/. They reported an accuracy of 93%. However, due to the use of K-fold cross-validation and recording multiple instances from the same participant, their findings are not generalizable.

In this example, we aimed to develop acoustic-based hypertension screening models using covert speech from 573 participants (197 women). The speech corpora, consisting of words and short sentences, were different for each participant, as they were asked to read randomly selected sentences during multiple recordings over the course of two weeks. We employed two different hypertension criteria for labeling each participant. We proposed three different frameworks for the classification task using 12 different classifiers. To ensure the robustness of the results and the generalizability of the findings, we evaluated the proposed models using a leave-one-subject-out cross-validation approach.

### Methods

### Recruitment and Dataset

Participants were enrolled from a broader example investigating the impact of glucose levels on vocal characteristics (Clinical Trial Identifier: CTRI/2021/08/035957), where all participants signed a written informed consent. Blood pressure and heart rate were measured at the example's start and again at the end of the two-week data collection period. Blood pressure was recorded with participants in a seated position after resting and was collected using standard procedure by qualified medical professionals (69). Additionally, age, height, weight, and BMI were recorded. All participants were non-smokers.

Participants were asked to read a randomly selected sentence from a list of approximately 3000 short English sentences, which they performed up to six times daily over the two weeks using a custom mobile application. Each time the participants started a new recording session a new random sentence was selected from the list of pre-generated sentences. By doing so, participants had a very low likelihood of recording the same sentence twice. The list of unique sentences that were used by participants were generated from an online random sentence generator (70). Recordings were captured at a sampling frequency of 44,100 Hz, anonymized, and stored on a secure cloud server accessible only via a customized API key. Each recording was transcribed, and only recordings that aligned with the provided random sentence were included in the analysis. No identifiable information was included in the voice recordings.

### Label Annotation

There are different clinical guidelines for annotating a hypertensive blood pressure recording. In this example we rely on two different criteria: 1) SBP≥140 mmHg OR DBP≥90 mmHg, which is selected based on European Society of Hypertension (ESH) hypertension guideline (63), and 2) SBP≥135 mmHg OR DBP≥85 mmHg, which is selected to address the imbalance number of hypertensive and non-hypertensive samples and derived from the average of the American Hypertension Association (AHA) and ESH guidelines for hypertension (63, 64).

### Feature Extraction

Four main categories of features were extracted from the speech recordings: 1) non-linear, 2) low-level descriptors (LLDs), 3) time-related, and 4) frequency-related.

Non-linear Features may include Shannon and logarithm entropies.

Low-level Descriptors (LLDs) may include the first four statistical moments (mean, variance, kurtosis, and skewness), the range (difference between peak and valley of the recording), and the cumulative sum of the first derivative (CSFD) of the signal, defined as Equation (3), where x[n] is the input speech recording of length N:

$$CSFD = {\sum }_{i = 2}^{N} x [ i ] - x [ i - 1 ] \qquad Equation ( 3 )$$

Time-related Features may include the mean and standard deviation of the zero-crossing rate over different frames of the input signal.

Frequency-related Features may include the mean and standard deviation of 13 Mel-frequency cepstral coefficients (MFCCs), ΔMFCCs, and ΔΔMFCCs, mean and standard deviation of 12 chromagram coefficients, mean and standard deviation of spectral contrast (7 coefficients), centroid, bandwidth, flatness, and roll-off, the fundamental frequency, LLDs of the tempogram, six equidistant spanned power spectral density (PSD) from 0 to

$$\frac{{F}_{s}}{2} Hz ,$$

and LLDs and entropies of PSD, as well as the third coefficients of the polynomial curve fitted to the PSD.

The spectral features from the frequency-related subset were extracted using a Hanning window with a size of 2048 samples and a hop size of 512 samples. The PSD was generated using the Welch method with a Hanning window size of 100 ms (equivalent to 4410 samples) and a 75% overlap. The frequency resolution of the PSD was set to four times greater than the window size. The fusion of all the feature subsets mentioned above would result in a feature dimension of 160.

### Frameworks

In this example, we explored the use of speech features in three different frameworks:
Whole Recording, where features were extracted from the entire speech recording.
Quarter-Based, where each recording was divided into four equidistant quarters, and features were extracted from each quarter.
Stacked-Quarter, where features from all four quarters were concatenated to form a comprehensive feature matrix, resulting in 640 features.

For each framework, the average of the features may be computed per subject, providing a unique feature vector for each participant. The length of these vectors was either 160 or 640, depending on the framework, and these vectors were used in subsequent stages of model development.

### Feature Selection

To reduce the dimensionality of the features used in model development and mitigate the risk of overfitting, we employed a two-step feature selection process. This process involved the use of two algorithms: Searching for Uncorrelated List of Variables (SULOV) (65) and Least Absolute Shrinkage and Selection Operator (LASSO) (66). First, SULOV was applied to remove features highly correlated with other features, with a correlation threshold set at 0.8. Then, LASSO was used to penalize less contributory features in a linear regression model, with the a parameter set at 0.01, unless specified otherwise.

For selecting the optimum set of features, 80% of the data, randomly shuffled in each iteration, was used to run the feature selection models. This procedure was repeated for 5 iterations. Features that appeared in at least three iterations were selected for the final model development stage. The number of selected features is denoted as Nₛ in the remaining sections.

### Model Development

Since the data in both criteria are highly imbalanced, we used the synthetic minority oversampling technique (SMOTE). We used seven nearest neighbors of each sample in both hypertensive and non-hypertensive groups to create an approximately balanced set of data. This technique was only applied to the training set of data and not validation and/or test sets to reduce the chance of data leakage for fair evaluation and better generalizability of the findings.

For predictive model development, we utilized 12 different classifiers: support vector classifier (SVC) and its extended version NuSVC, K-nearest neighbors (KNN), logistic regression (LR), linear discriminant analysis (LDA), quadratic discriminant analysis (QDA), Gaussian naïve Bayes (GNB), Bernoulli naïve Bayes (BNB), Ridge, extreme gradient boosting (XGB), decision tree (DT), and random forest (RF). The default parameter values from the Scikit-learn Python library were used. Given the distinct patterns between men and women, separate models were trained for each sex. The best-performing classifier for each sex is reported in the results section.

### Evaluation

A leave-one-subject-out (LOSO) approach was employed for model development and performance evaluation. In this approach, the feature vector of each participant is left out as the test set, while the remaining feature vectors form the training feature matrix. This procedure is repeated for all participants. To measure the performance of the different models, we used balanced accuracy (BACC), which is the arithmetic average of sensitivity and specificity, and the area under the receiver operating characteristic curve (AuROC). Additionally, to further visualize the performance of each model, ROC curves were plotted.

### Implementation Configurations

Speech features were extracted using Numpy (version 1.26.3) and Librosa (version 0.10.1) (67) libraries. All machine learning models, and feature selection techniques were implemented using the Scikit-learn package (version 1.2.2) (68).

### Results

### Participant Demographics

In total, 573 participants completed the research protocol of which 197 were female. The measurements were assessed based on two criteria: SBP≥135 mmHg or DBP≥85 mmHg, and SBP≥140 mmHg or DBP≥90 mmHg. The details for these criteria are presented in FIGS. 15 and 16, respectively. Upon comparing the tables, it can be inferred that the stricter criterion (SBP≥140 mmHg or DBP≥90 mmHg) results in a smaller number of participants classified as hypertensive, consequently leading to a more imbalanced dataset, which justified the inclusion of the more relaxed hypertension guideline.

### Whole Recording

The performance of the predictive models based on the features extracted from the whole recording is shown in FIGS. 17 and 20. As indicated by the table, the models performed better in terms of BACC for females compared to males. This improved performance can be attributed to the smaller number of female participants, resulting in a simpler but less generalizable model. Additionally, for females, the model performance was better under the more relaxed threshold. Conversely, the stricter threshold yielded the best result for males, with a BACC of 0.65.

### Quarter-Based Performance

The classification results for the proposed quarter-based pipeline in both hypertension criteria are presented in FIGS. 18 and 21. The table reveals that that male models based on the third and fourth quarter demonstrate higher performance compared to other quarters. For female models, the first and fourth quarters exhibit the best performance in BACC. Notably, the fourth quarter for females using the more relaxed criterion and the fourth quarter for males using the stricter criterion yielded improved model performance compared to the models developed using the entire speech recording (Females: BACC 0.7, AuROC 0.68; Males: BACC 0.65, AuROC 0.70).

### Stacked Quarters

Integrating features from all quarters, as depicted in FIGS. 19 and 22, achieved the highest performance, with a BACC of 0.71 and AuROC of 0.72 for males, and a BACC of 0.78 and AuROC of 0.78 for females. Given that this model is based on a large number of features (640 dimensions), the remaining feature set after dimensionality reduction is still noticeably high (˜100 features), raising potential concerns about over-fitting. However, the LOSO approach employed in the model development and evaluation procedures mitigates such risks. Additionally, it is worth noting that Ridge and LDA classifiers were selected for this framework, both of which are well-suited for handling high-dimensional classification tasks.

### Discussion

In this example, we introduced three different frameworks for screening hypertension from speech recordings based on randomly selected English sentences from a relatively large cohort. Throughout the investigated frameworks, we demonstrated the dynamics of speech by splitting the recordings into four equidistant quarters and developing predictive models based on different combinations of such segments.

Our example, for the first time, introduced a predictive model based on covert speech instead of vowels and/or fixed sentence recording protocols. This advancement may bring speech-based hypertension screening one step closer to real-world applications. By splitting the recordings into quarters, we improved analysis resolution to capture hypertension-related differences across different parts of the recordings. Furthermore, by employing the LOSO cross validation approach, common inconsistencies were addressed in reporting validation performance such as the omission of confidence intervals and demonstrated the generalizability of the proposed method.

Although this approach might seem non-intuitive due to the recording of random sentences that may exhibit varying vocal characteristics, merging them using the mean operator results in a unique representation of the hypertension-related acoustic vector for each participant. This vector could reflect the hypertension level of each participant.

Our quarter-based analysis revealed higher levels of hypertension-related information in the second and third quarters of the recordings. This suggests that the first and last quarters, which correspond to the start and end of the recordings, are more likely to contain uncertainty and less stability in sentence articulation. Consequently, the middle segments of speech carry more hypertension-related information, making them more suitable for model development.

### Conclusion

This example investigated the feasibility of using acoustic representations of random speech recordings for hypertension screening. Utilizing two different clinically supported hypertension guidelines, we demonstrated that our best proposed framework achieved a BACC of 61% for males and 70% for females under the relaxed criterion (SBP≥135 OR DBP≥85), and a BACC of 71% for males and 78% for females under the stricter EHS guidelines (SBP≥140 OR DBP≥90). The findings of this example suggest the potential of using covert speech for hypertension screening.

### REFERENCES

1. Group, S. R. A randomized trial of intensive versus standard blood-pressure control. New Engl. J. Medicine 373, 2103-2116 (2015).
2. Forouzanfar, M. et al. Oscillometric blood pressure estimation: past, present, and future. IEEE reviews biomedical engineering 8, 44-63 (2015).
3. WHO. Global report on hypertension: The race against a silent killer (2023).
4. Fuchs, F. D. & Whelton, P. K. High blood pressure and cardiovascular disease. Hypertension 75, 285-292 (2020).
5. Di Palo, K. E. & Barone, N. J. Hypertension and heart failure: prevention, targets, and treatment. Hear. failure clinics 16, 99-106 (2020).
6. Ritz, E. Hypertension and kidney disease. Clin. nephrology 74, S39-43 (2010).
7. Escobar, E. Hypertension and coronary heart disease. J. human hypertension 16, S61-S63 (2002).
8. Grossman, E. & Messerli, F. H. Hypertension and diabetes. Cardiovasc. diabetology: Clin. metabolic inflammatory facets 45, 82-106 (2008).
9. Lovibond, K. et al. Cost-effectiveness of options for the diagnosis of high blood pressure in primary care: a modelling study. The lancet 378, 1219-1230 (2011).
10. Hasanzadeh, N., Ahmadi, M. M. & Mohammadzade, H. Blood pressure estimation using photoplethysmogram signal and its morphological features. IEEE Sensors J. 20, 4300-4310 (2019).
11. Kachuee, M., Kiani, M. M., Mohammadzade, H. & Shabany, M. Cuffless blood pressure estimation algorithms for continuous health-care monitoring. IEEE Transactions on Bio-med. Eng. 64, 859-869 (2016).
12. Reddy, M. K. et al. The automatic detection of heart failure using speech signals. Comput. Speech & Lang. 69, 101205 (2021).
13. Amir, O. et al. Remote speech analysis in the evaluation of hospitalized patients with acute decompensated heart failure. Hear. Fail. 10, 41-49 (2022).
14. Murton, O. M. et al. Acoustic speech analysis of patients with decompensated heart failure: a pilot study. The J. Acoust. Soc. Am. 142, EL401-EL407 (2017).
15. Priyasad, D. et al. Detecting heart failure through voice analysis using self-supervised mode-based memory fusion. In Proceedings of the 23rd INTERSPEECH Conference, 2848-2852 (International Speech Communication Association, 2022).
16. R. Saloni, K. Sharma, and A. K. Gupta, “Classification of high blood pressure persons vs normal blood pressure persons using voice analysis,” IJ Image, Graphics and Signal Processing, pp. 47-52, 2014.
17. M. Sakai, “Feasibility study on blood pressure estimations from voice spectrum analysis,” International Journal of Computer Applications, vol. 109, no. 7, pp. 39-43, 2015.
18. Ankishan, H. Blood pressure prediction from speech recordings. Biomed. Signal Process. Control. 58, 101842 (2020).
19. Kaufman, J. M., Thommandram, A. & Fossat, Y. Acoustic analysis and prediction of type 2 diabetes mellitus using smartphone-recorded voice segments. Mayo Clin. Proceedings: Digit. Heal. 1, 534-544 (2023).
20. C. Cuspidi, M. Tadic, G. Grassi, and G. Mancia, “Treatment of hypertension: The esh/esc guidelines recommendations,” Pharmacological research, vol. 128, pp. 315-321, 2018.
21. J. M. Flack and B. Adekola, “Blood pressure and the new acc/aha hypertension guidelines,” Trends in cardiovascular medicine, vol. 30, no. 3, pp. 160-164, 2020.
22. Chawla, N. V., Bowyer, K. W., Hall, L. O. & Kegelmeyer, W. P. Smote: synthetic minority over-sampling technique. J. artificial intelligence research 16, 321-357 (2002).
23. McFee, B. et al. librosa: Audio and music signal analysis in python. In Proceedings of the 14th python in science conference, vol. 8, 18-25 (2015).
24. Pedregosa, F. et al. Scikit-learn: Machine learning in python. J. machine learning research 12, 2825-2830 (2011).
25. Sara, J. D. S. S. et al. Non-invasive vocal biomarker is associated with pulmonary hypertension. Circulation 140, A12549-A12549 (2019).
26. Alvear, R. M. B. d., Barón-López, F. J., Alguacil, M. D. & Dawid-Milner, M. S. Interactions between voice fundamental frequency and cardiovascular parameters. preliminary results and physiological mechanisms. Logop. Phoniatrics Vocology 38, 52-58 (2013).
27. LYNCH, J. J. et al. Human speech and blood pressure. The J. nervous mental disease 168, 526-534 (1980).
28. Byeon, H. & Cha, S. Evaluating the effects of smoking on the voice and subjective voice problems using a meta-analysis approach. Sci. Reports 10, 4720 (2020).
29. Primatesta, P., Falaschetti, E., Gupta, S., Marmot, M. G. & Poulter, N. R. Association between smoking and blood pressure: evidence from the health survey for england. Hypertension 37, 187-193 (2001).
30. Yao, X., Jitsuhiro, T., Miyajima, C., Kitaoka, N. & Takeda, K. Physical characteristics of vocal folds during speech under stress. In 2012 IEEE International Conference on Acoustics, Speech and Signal Processing (ICASSP), 4609-4612 (IEEE, 2012).
31. Kulkarni, S., O'Farrell, I., Erasi, M. & Kochar, M. Stress and hypertension. WMJ: official publication State Med. Soc. Wis. 97, 34-38 (1998).
32. Da Cunha, M. G. B., Passerotti, G. H., Weber, R., Zilberstein, B. & Cecconello, I. Voice feature characteristic in morbid obese population. Obes. surgery 21, 340-344 (2011).
33. Seravalle, G. & Grassi, G. Obesity and hypertension. Pharmacol. research 122, 1-7 (2017).
34. Stogowska, E., Kaminski, K. A., Ziółko, B. & Kowalska, I. Voice changes in reproductive disorders, thyroid disorders and diabetes: a review. Endocr. Connect. 11 (2022).
35. Berta, E. et al. Hypertension in thyroid disorders. Front. endocrinology 10, 457798 (2019).
36. Lago, R. M., Singh, P. P. & Nesto, R. W. Diabetes and hypertension. Nat. clinical practice Endocrinol. & metabolism 3, 667-668 (2007).
37. Ravi, R. & Gunjawate, D. R. Effect of diabetes mellitus on voice: a systematic review. Pract. Diabetes 36, 177-180 (2019).
38. Konecny, T., Kara, T. & Somers, V. K. Obstructive sleep apnea and hypertension: an update. Hypertension 63, 203-209 (2014).
39. Davidson, T. M. The great leap forward: the anatomic basis for the acquisition of speech and obstructive sleep apnea. Sleep medicine 4, 185-194 (2003).
40. Hari Kumar K V S, Garg A, Ajai Chandra N S, Singh S P, Datta R. Voice and endocrinology. Indian J. Endocrinol. Metab. 2016. DOI:10.4103/2230-8210.190523.
41. Noffs G, Perera T, Kolbe S C, et al. What speech can tell us: A systematic review of dysarthria characteristics in Multiple Sclerosis. Autoimmun. Rev. 2018. DOI:10.1016/j.autrev.2018.06.010
42. Maor E, Perry D, Mevorach D, et al. Vocal Biomarker Is Associated With Hospitalization and Mortality Among Heart Failure Patients. J Am Heart Assoc 2020. DOI:10.1161/JAHA.119.013359.
43. Vaiciukynas E, Verikas A, Gelzinis A, Bacauskiene M. Detecting Parkinson's disease from sustained phonation and speech signals. PLoS One 2017.

DOI:10.1371/journal.pone.0185613.
44. Fraser K C, Meltzer J A, Rudzicz F. Linguistic features identify Alzheimer's disease in narrative speech. J Alzheimer's Dis 2015. DOI:10.3233/JAD-150520.
45. Marmar C R, Brown A D, Qian M, et al. Speech-based markers for posttraumatic stress disorder in US veterans. Depress Anxiety 2019. DOI:10.1002/da.22890.
46. Bonneh Y S, Levanon Y, Dean-Pardo O, Lossos L, Adini Y. Abnormal speech spectrum and increased pitch variability in young autistic children. Front Hum Neurosci 2011. DOI:10.3389/fnhum.2010.00237.
47. B. Zhou et al., “Worldwide trends in blood pressure from 1975 to 2015: a pooled analysis of 1479 population-based measurement studies with 19-1 million participants,” The Lancet, vol. 389, no. 10064, pp. 37-55, 2017.
48. B. M. Egan, Y. Zhao, and R. N. Axon, “US trends in prevalence, awareness, treatment, and control of hypertension, 1988-2008,” Jama, vol. 303, no. 20, pp. 2043-2050, 2010.
49. T. Beaney et al., “May Measurement Month 2019: the global blood pressure screening campaign of the International Society of Hypertension,” Hypertension, vol. 76, no. 2, pp. 333-341, 2020.
50. W. Viechtbauer, “Bias and efficiency of meta-analytic variance estimators in the random-effects model,” J. Educ. Behav. Stat., vol. 30, no. 3, pp. 261-293, 2005.
51. C. K. Chow et al., “Prevalence, awareness, treatment, and control of hypertension in rural and urban communities in high-, middle-, and low-income countries,” Jama, vol. 310, no. 9, pp. 959-968, 2013.
52. V. J. Dzau and C. A. Balatbat, “Future of hypertension: The need for transformation,” Hypertension, vol. 74, no. 3, pp. 450-457, 2019.
53. D. Priyasad et al., “Detecting heart failure through voice analysis using self-supervised mode-based memory fusion,” presented at the Proceedings of the 23rd INTERSPEECH Conference, International Speech Communication Association, 2022, pp. 2848-2852.
54. O. M. Murton et al., “Acoustic speech analysis of patients with decompensated heart failure: a pilot study,” J. Acoust. Soc. Am., vol. 142, no. 4, pp. EL401-EL407, 2017.
55. O. Amir et al., “Remote speech analysis in the evaluation of hospitalized patients with acute decompensated heart failure,” Heart Fail., vol. 10, no. 1, pp. 41-49, 2022.
56. M. K. Reddy et al., “The automatic detection of heart failure using speech signals,” Comput. Speech Lang., vol. 69, p. 101205, 2021.
57. J. M. Kaufman, A. Thommandram, and Y. Fossat, “Acoustic analysis and prediction of type 2 diabetes mellitus using smartphone-recorded voice segments,” Mayo Clin. Proc. Digit. Health, vol. 1, no. 4, pp. 534-544, 2023.
58. Y. Fossat and J. Jeon, “Systems and methods for generating models for determining blood glucose levels using voice,” January 2024.
59. E. Goldshtein, A. Tarasiuk, and Y. Zigel, “Automatic detection of obstructive sleep apnea using speech signals,” IEEE Trans. Biomed. Eng., vol. 58, no. 5, pp. 1373-1382, 2010.
60. J. Robin, J. E. Harrison, L. D. Kaufman, F. Rudzicz, W. Simpson, and M. Yancheva, “Evaluation of speech-based digital biomarkers: review and recommendations,” Digit. Biomark., vol. 4, no. 3, pp. 99-108, 2020.
61. H. Ankişhan, “Blood pressure prediction from speech recordings,” Biomed. Signal Process. Control, vol. 58, p. 101842, 2020.
62. B. Taghibeyglou, J. Kaufman, and Y. Fossat, “Machine Learning-enabled Hypertension Screening through Acoustical Speech Analysis: Model Development and Validation,” IEEE Access.
63. G. Mancia et al., “2023 ESH Guidelines for the management of arterial hypertension The Task Force for the management of arterial hypertension of the European Society of Hypertension: Endorsed by the International Society of Hypertension (ISH) and the European Renal Association (ERA),” J. Hypertens., vol. 41, no. 12, 2023, [Online]. Available: https://journals.lww.com/jhypertension/fulltext/2023/12000/2023_esh_guidelines_for the_management_of_arterial.2.aspx
64. J. M. Flack and B. Adekola, “Blood pressure and the new ACC/AHA hypertension guidelines,” Trends Cardiovasc. Med., vol. 30, no. 3, pp. 160-164, April 2020, doi: 10.1016/j.tcm.2019.05.003.
65. A. Unler, A. Murat, and R. B. Chinnam, “mr2PSO: A maximum relevance minimum redundancy feature selection method based on swarm intelligence for support vector machine classification,” Inf. Sci., vol. 181, no. 20, pp. 4625-4641, October 2011, doi: 10.1016/j.ins.2010.05.037.
66. R. Muthukrishnan and R. Rohini, “LASSO: A feature selection technique in predictive modeling for machine learning,” in 2016 IEEE International Conference on Advances in Computer Applications (ICACA), October 2016, pp. 18-20. doi: 10.1109/ICACA.2016.7887916.
67. B. McFee et al., “librosa: Audio and Music Signal Analysis in Python,” presented at the Python in Science Conference, Austin, Texas, 2015, pp. 18-24. doi: 10.25080/Majora-7b98e3ed-003.
68. F. Pedregosa et al., “Scikit-learn: Machine learning in Python,” J. Mach. Learn. Res., vol. 12, pp. 2825-2830, 2011.
69. O. John et al., The 2020 “WHO technical specifications for automated non-invasive blood pressure measuring devices with cuff”. Hypertension. 2021 Mar. 3; 77(3):806-12.
70. “Random Sentence Generator.” The Word Finder. thewordfinder.com. https://www.thewordfinder.com/random-sentence-generator/.

We claim:

1. A computer-implemented method for generating a hypertension indicator prediction model, the method comprising:
providing, at a memory:
a plurality of voice samples from at least one subject at a plurality of time points; and
a plurality of blood pressure measurements, wherein each blood pressure measurement comprises a diastolic measurement and a systolic measurement, and each blood pressure measurement is associated with a corresponding subject in the at least one subject;
sorting, at a processor in communication with the memory, the plurality of voice samples into two or more hypertensive categories based on the blood pressure measurement;
extracting, at the processor, voice feature values for a set of voice features from each of the plurality of voice samples;
selecting, at the processor, a subset of voice features from the set of voice features; and
generating at the processor, the hypertension indicator prediction model based on the subset of voice features.

2. The method of claim 1, wherein generating the hypertension indicator prediction model based on the subset of voice features comprises determining a weight for each voice feature in the subset of voice features.

3. The method of claim 2, wherein the method further comprises:
selecting, at the processor, a subset of voice features from the set of voice features based on a Least Absolute Shrinkage and Selection Operator (LASSO) method.

4. The method of claim 3 wherein the LASSO objective function is
$${min}_{β} \frac{1}{2 N} {‖ X β - y ‖}_{2}^{2} + α {‖ β ‖}_{1} .$$

5. The method of claim 4, wherein each of the voice features are selected from the group of a non-linear feature, a low-level descriptor feature, a time related feature, and a frequency-related feature.

6. The method of claim 5, wherein:
the non-linear feature comprises one of a Shannon entropy of the voice sample and a logarithm entropy of the voice sample,
the low-level descriptor feature comprises one of a mean of the voice sample, a variance of the voice sample, a skewness of the voice sample, a kurtosis of the voice sample, a range of the voice sample, a cumulative sum of the first derivative (CSFD) of the signal,
the time related feature comprises one of a mean of the zero-crossing rate, a standard deviation of the zero-crossing rate, and
the frequency-related feature comprises one of a mean Mel-scale frequency cepstral coefficient (MFCC), a standard deviation of a Mel-scale frequency cepstral coefficient (MFCC), a first (ΔMFCC) and a second derivative (ΔΔMFCC) of the MFCC, a mean Chromagram coefficient, a standard deviation of a Chromagram coefficient, a mean spectral contrast, a standard deviation of spectral contrast, a centroid of a spectral contrast, a bandwidth of a spectral contrast, a flatness of a spectral contrast, a roll-off of a spectral contrast, a fundamental frequency, an LLD of a tempogram, a power spectral density (PSD) from 0 to
$$\frac{{F}_{s}}{2} Hz ,$$
an LLD of the PSD, an entropies of the PSD, and a coefficient of a polynomial curve fitted to the PSD.

7. The method of claim 1, further comprising:
dividing, at the processor, each of the plurality of voice samples a plurality of equidistant segments;
extracting, at the processor, voice feature values for a set of voice features from each of the plurality of equidistant segments; and
wherein the voice feature values for each voice sample comprise a concatenation of the voice feature values for the corresponding plurality of equidistant segments.

8. The method of claim 7, further comprising:
averaging, at the processor, at least two voice samples in the plurality of voice samples, the at least two voice samples corresponding to the same subject;
applying, at the processor, a synthetic minority oversampling technique (SMOTE) to increase a number of the plurality of voice samples; and
determining, at the processor, a true positive rate (TPR) and a true negative rate (TNR) for the generated hypertension indicator prediction model using a leave-one-subject-out (LOSO) method.

9. The method of claim 8, wherein the two or more hypertensive categories comprise at least a first hypertensive category comprising SBP>=135 OR DBP>=85 and a second hypertensive category comprising SBP>=140 OR DBP>=90.

10. The method of claim 9, wherein each of the plurality of voice samples comprises: a predetermined phrase vocalized by the at least one subject or a randomly selected sentence vocalized by the subject.

11. A system for generating a hypertension indicator prediction model, the system comprising:
a memory comprising:
a plurality of voice samples from at least one subject at a plurality of time points; and
a plurality of blood pressure measurements, wherein each blood pressure measurement comprises a diastolic measurement and a systolic measurement, and each blood pressure measurement is associated with a corresponding subject in the at least one subject;
a processor in communication with the memory, the processor configured to:
sorting the plurality of voice samples into two or more hypertensive categories based on the blood pressure measurement;
extracting, at the processor, voice feature values for a set of voice features from each of the plurality of voice samples;
selecting, at the processor, a subset of voice features from the set of voice features; and
generating at the processor, the hypertension indicator prediction model based on the subset of voice features.

12. The system of claim 11, wherein generating the hypertension indicator prediction model based on the subset of voice features comprises determining a weight for each voice feature in the subset of voice features.

13. The system of claim 12, wherein the processor is further configured to:
select a subset of voice features from the set of voice features based on a Least Absolute Shrinkage and Selection Operator (LASSO) method.

14. The system of claim 13 wherein the LASSO objective function is
$${min}_{β} \frac{1}{2 N} {‖ X β - y ‖}_{2}^{2} + α {‖ β ‖}_{1} .$$

15. The system of claim 14, wherein each of the voice features are selected from the group of a non-linear feature, a low-level descriptor feature, a time related feature, and a frequency-related feature.

16. The system of claim 15, wherein:
the non-linear feature comprises one of a Shannon entropy of the voice sample and a logarithm entropy of the voice sample,
the low-level descriptor feature comprises one of a mean of the voice sample, a variance of the voice sample, a skewness of the voice sample, a kurtosis of the voice sample, a range of the voice sample, a cumulative sum of the first derivative (CSFD) of the signal,
the time related feature comprises one of a mean of the zero-crossing rate, a standard deviation of the zero-crossing rate, and
the frequency-related feature comprises one of a mean Mel-scale frequency cepstral coefficient (MFCC), a standard deviation of a Mel-scale frequency cepstral coefficient (MFCC), a first (ΔMFCC) and a second derivative (ΔΔMFCC) of the MFCC, a mean Chromagram coefficient, a standard deviation of a Chromagram coefficient, a mean spectral contrast, a standard deviation of spectral contrast, a centroid of a spectral contrast, a bandwidth of a spectral contrast, a flatness of a spectral contrast, a roll-off of a spectral contrast, a fundamental frequency, an LLD of a tempogram, a power spectral density (PSD) from 0 to
$$\frac{{F}_{s}}{2} Hz ,$$
an LLD of the PSD, an entropies of the PSD, and a coefficient of a polynomial curve fitted to the PSD.

17. The system of claim 11, wherein the processor is further configured to:
divide each of the plurality of voice samples a plurality of equidistant segments;
extract voice feature values for a set of voice features from each of the plurality of equidistant segments; and
wherein the voice feature values for each voice sample comprise a concatenation of the voice feature values for the corresponding plurality of equidistant segments.

18. The system of claim 17, wherein the processor is further configured to:
average at least two voice samples in the plurality of voice samples, the at least two voice samples corresponding to the same subject;
apply a synthetic minority oversampling technique (SMOTE) to increase a number of the plurality of voice samples; and
determine a true positive rate (TPR) and a true negative rate (TNR) for the generated hypertension indicator prediction model using a leave-one-subject-out (LOSO) method.

19. The system of claim 11, wherein the two or more hypertensive categories comprise at least a first hypertensive category comprising SBP>=135 OR DBP>=85 and a second hypertensive category comprising SBP>=140 OR DBP>=90.

20. The system of claim 11, wherein each of the plurality of voice samples comprises: a predetermined phrase vocalized by the at least one subject or a randomly selected sentence vocalized by the subject.
